(19) United States
(12) Patent Application Publication
(10) Pub. No.: US 2015/0100024 A1
(43) Pub. Date: Apr. 9, 2015

(54) MEDICAMENT DELIVERY AND TRAINING CARTRIDGE SYSTEM AND MECHANISMS OF ACTUATION

(71) Applicants: Jeff Baker, Orlando, FL (US); Mark Bunker, Orlando, FL (US); Paul van der Pol, Winter Garden, FL (US); Seth Freytag, Winter Springs, FL (US); Christopher Chung, Orlando, FL (US); Andrew Palmer, Orlando, FL (US); Francis Michael Siemer, Orlando, FL (US); Craig Baker, Lebanon, OH (US)

(72) Inventors: Jeff Baker, Orlando, FL (US); Mark Bunker, Orlando, FL (US); Paul van der Pol, Winter Garden, FL (US); Seth Freytag, Winter Springs, FL (US); Christopher Chung, Orlando, FL (US); Andrew Palmer, Orlando, FL (US); Francis Michael Siemer, Orlando, FL (US); Craig Baker, Lebanon, OH (US)

(21) Appl. No.: 14/505,844

(22) Filed: Oct. 3, 2014

Related U.S. Application Data
(60) Provisional application No. 61/886,596, filed on Oct. 3, 2013.

(51) Int. Cl. A61M 5/20; G09B 23/28

(52) U.S. Cl. CPC A61M 5/2033; G09B 23/285; A61M 2005/2026; A61M 2005/206
USPC 604/157; 434/262

(57) ABSTRACT

A medicament delivery system including a container for storing a medicament prior to use is provided. The container includes one or more openings therein, a first housing for containing a medicament, a stopper, a second housing, wherein the first housing is held within the second housing and the first housing is movable relative to the second housing. The system further includes an injection member a spring disposed between a lower portion of the first housing and a lower portion of the second housing, wherein an actuation mechanism interacts with the container to eject the injection member from the second housing, and the stopper moves relative to the first housing in a first direction to deliver the medicament through the injection member, and following injection, the injection member is retracted into the second housing to prevent an unintentional contact with the injection member.

### CROSS-REFERENCE TO RELATED APPLICATIONS

This application claims priority to and incorporates by reference the contents of Provisional Application No. 61/886,596 filed on Oct. 3, 2013.

### BACKGROUND

Manual disposable syringe based devices have existed since the mid-1800's. These devices were designed for a single purpose of performing a subcutaneous injection through a hollow-bore needle affixed to the syringe device. Syringes are simple mechanical systems with no capability of refined fluid dynamics or ability to integrate advanced digital capabilities.

Auto-injection or “pen” devices have recently become increasingly popular for single dose or multi dose, at home self-administration. These auto-injection devices are primarily designed to accomplish two basic objectives: convenience and automation of drug delivery in an outpatient setting. These are typically mechanically spring-loaded devices that advance a component of the device to transfer medication via hollow-bore needle to a patient's tissues.

Auto-injection devices lack the ability to regulate whether the medication is actually delivered to the patient or whether it is delivered to a correct location. Most auto-injection devices fail to integrate advanced digital capabilities. A significant limitation is the inability of auto-injection devices to collect and transfer digital information from the device to other sources.

Injectable medications are required for a number of varying illnesses and diseases. A number of injectable medications require self-injection by a patient. Self-injection of a medicament using a device having a needle carries with it a certain stigma. Oftentimes patients are weary of injecting themselves for fear or anxiety related to failing to receive a complete dose of the medication, pain associated with injecting oneself with the needle, accidentally sticking oneself with the needle, and difficulties in adequately grasping the dosing mechanism to inject oneself, among other concerns.

Auto-injection devices are routinely used to provide a means for self-injecting certain medications. The size and operation of these auto-injection devices can often be daunting to a patient, whether they are injecting themselves for the first or they have injected themselves before. These fears and anxieties associated with the currently available self-injection devices, particularly the auto-injection devices, may result in the administration of an incomplete dose of a medicament, failure to administer any portion of the dose of a medicament, or accidentally sticking oneself with the needle of the device, which in some instances could lead to unwanted transmission of diseases if the needle is contaminated.

In some instances, after an auto-injection is complete, the contaminated needle is retracted within the auto-injection device or covered over by a needle guard or sheath and the entire auto-injection device is disposed of. Therefore, most auto-injectors currently available are single use auto-injectors. These single use auto-injectors are costly and economically wasteful. Alternatively, there are injection devices which require a user to re-cap a needle after the injection is complete such that the disposable needle can be removed and discarded. These injection devices carry with them the risk of unwanted sticking of oneself during re-capping of the needle.

An additional concern exists with regard to injection devices, and in particular with regard to auto-injectors, where users with little or no medical knowledge or experience are injecting themselves or injecting others using these devices. Performing a medical treatment or test on oneself or others carries with it certain risks and often creates a level of anxiety for the user performing the treatment or test. It has proven beneficial in the medical field to practice various medical techniques including drug delivery, specifically where it relates to injections and other invasive drug delivery means prior to delivering the medications to a patient in need, and particularly in the case of self-administration of medicaments. Training devices are helpful in reducing anxiety associated with self administering medical treatment, as well as increasing efficiency and accuracy in providing the treatment to patients. Medical devices can be intimidating to use; the fear associated with giving oneself an injection, for example, can be traumatic. This fear is increased in persons with little or no experience in self-administration of medications. Consequently, devices to assist in training individuals to inject themselves or otherwise self-administer medication are beneficial in decreasing or preventing the anxiety associated with medicament delivery.

Therefore, there exists a need for an injection device which may be safely and efficiently used by patients without medical experience in preparing and self-injecting medications. Furthermore, a device which closely resembles a medicament delivery device that can be used to simulate an injection for training purposes would be highly beneficial.

### SUMMARY

In an embodiment, a medicament delivery system including a container for storing a medicament prior to use is provided. The container may include one or more openings therein, a first housing for containing a medicament, a stopper, a second housing, wherein the first housing is held within the second housing and the first housing is movable relative to the second housing. The system may further include an injection member, a spring disposed between a lower portion of the first housing and a lower portion of the second housing, wherein an actuation mechanism interacts with the container to eject the injection member from the second housing, and the stopper moves relative to the first housing in a first direction to deliver the medicament through the injection member, and following injection, the injection member is retracted into the second housing to prevent an unintentional contact with the injection member.

In another embodiment, a medicament delivery training system configured to provide stepwise instructions for using the system to a user in a particular sequence is provided. The system includes a reusable housing component configured to receive a container, the reusable housing component including a control interface, the control interface including at least one responsive member reactive to a user input, an actuation mechanism configured to interact with the container, a signal output component associated with the reusable housing component, and circuitry associated with the reusable housing component configured to control a provision of the stepwise instructions to the user in the particular sequence. The container comprises a first housing, and a second housing, a spring disposed between a lower portion of the first housing and a lower portion of the second housing, and an injection member associated with the lower portion of the first housing, wherein the first housing is movable relative to the second housing; and a stopper is associated with the first housing, the stopper being movable relative to the first housing, wherein the actuation mechanism interacts with the container to move the first housing in a first direction relative to the second housing to eject the injection member from the second housing, and the stopper in a first direction relative to the first housing.

In a further embodiment, a medicament delivery system housing for use with a removable medicament container is provided. The housing includes (i) a generally ovoid shaped head portion, (ii) a shaft portion comprising a top end and bottom end, the top end associated with the head portion, (iii) a base associated with the bottom end of the shaft portion the base being wider than the shaft portion, (iv) a socket configured to receive the container, (v) an actuation button on said generally ovoid shaped head portion, (vi) an ejection mechanism for releasing the container when situated within the housing; and (vii) an actuation mechanism associated with the actuation button, said actuation mechanism configured to deliver medicament when the container is received within the housing and the actuation button is activated.

In a further embodiment, a medicament delivery system housing for use with a removable medicament container is provided. The housing includes a generally cylindrically shaped head portion comprising a first end and a second end having a smaller diameter than a middle section of the head portion, a shaft portion associated with the head portion, wherein in a resting position, an opening is disposed between at least a portion of the shaft portion and the head portion, a base associated with the shaft portion, the base provided for resting the housing on a surface, and an opening in the base configured to receive a container. The head portion may include an actuation button located at the first end thereof, the head and/or the shaft portion may include a compartment therein for receiving and/or storing a power source, a locking mechanism, wherein the locking mechanism is released when the shaft portion and the head portion are in contact with one another and the opening between the shaft portion and the head portion is removed, and wherein the housing includes an actuation mechanism configured to activate the container when the container is received within the housing, the actuation button is activated, and the locking mechanism is released.

In yet a further embodiment, a medicament delivery system housing for use with a removable medicament container is provided. The housing may include a generally oblong spherical shaped body, the body having an upper surface and a lower surface, the lower surface configured for resting on a user, the lower surface comprising an opening configured to receive a container, the upper surface comprising a deformable actuation mechanism component, the actuation mechanism component configured to activate the container when the component is deformed. The deformable actuation mechanism component may be configured for receiving a palm of a user, the body including an ejection mechanism configured to eject the container from the housing when the ejection mechanism is activated.

In still a further embodiment, a medicament delivery system housing for use with a removable medicament container is provided. The housing includes a generally cylindrically shaped body portion having a first end and a second end, wherein the first and second ends include a smaller diameter than a middle section of the body portion, and the second end includes an opening configured to receive a container. The head portion is associated with the body portion, wherein the head portion includes an upper surface and a lower surface, the head portion associates with the body portion at its lower surface, and wherein an actuation mechanism is disposed on its upper surface. The actuation mechanism is configured to activate the container, and the head portion further includes a release member configured to release the container upon activation of the release member.

In yet a further embodiment, a medicament delivery system is provided. The medicament delivery system includes a container for storing a medicament prior to use, the container including a first housing for containing a medicament, a stopper associated with the first housing, a second housing, wherein the first housing is held within the second housing, and the first housing is movable relative to the second housing, an injection member associated with a lower portion of the first housing, an actuation mechanism, wherein the actuation mechanism interacts with the container to move the first housing relative to the second housing in a first direction to eject the injection member from the second housing, traversing a first contaminant barrier, and move the stopper relative to the first housing in a first direction to deliver the medicament through the injection member, such that when the first housing moves relative to the second housing in a second direction, the injection member is retracted into the second housing to prevent an unintentional contact with the injection member.

In a further embodiment a container for storing a medicament prior to use is provided. The container may include a first housing for containing a medicament, a stopper associated with the first housing, a second housing, wherein the first housing is held within the second housing, and the first housing is movable relative to the second housing, and an injection member associated with a lower portion of the first housing, wherein an actuation mechanism is configured to interact with the container to move the first housing relative to the second housing in a first direction to eject the injection member from the second housing, traversing a first contaminant barrier, and move the stopper relative to the first housing in a first direction to deliver the medicament through the injection member, such that when the first housing moves relative to the second housing in a second direction, the injection member is retracted into the second housing to prevent an unintentional contact with the injection member.

In still a further embodiment, a container for storing a medicament prior to use is provided. The container includes a first housing for containing a medicament, a stopper associated with the first housing, a second housing, wherein the first housing is held within the second housing, and the first housing is movable relative to the second housing, an injection member associated with a lower portion of the first housing, the injection member including an injection member cap removably coupled thereto, and a cover surrounding at least a portion of the container, wherein the cap is affixed to the cover, and the cover includes a protrusion, wherein the protrusion is configured to engage the first housing and/or second housing to retain the cover on the container. An actuation mechanism is configured to interact with the container to move the first housing relative to the second housing in a first direction to eject the injection member from the second housing, and move the stopper relative to the first housing in a first direction to deliver the medicament through the injection member, such that when the first housing moves relative to the second housing in a second direction, the injection member is retracted into the second housing to prevent an unintentional contact with the injection member.

### BRIEF DESCRIPTION OF THE DRAWINGS

A more particular description briefly stated above will be rendered by reference to specific embodiments thereof that are illustrated in the appended drawings. Understanding that these drawings depict only typical embodiments and are not therefore to be considered to be limiting of its scope, the embodiments will be described and explained with additional specificity and detail through the use of the accompanying drawings in which:

FIG. 1 is a cross sectional view of a container according to an embodiment of the invention.

FIGS. 2A-2B are exploded views of the embodiment of the container shown in FIG. 1.

FIGS. 3A-3D are cross sectional views illustrating the steps of use of an embodiment of the medicament delivery system.

FIGS. 3A′-3C′ are cross sectional views illustrating the steps of use of another embodiment of the medicament delivery system.

FIGS. 4A-4D are cross sectional views of another embodiment of the medicament delivery system and illustrate the steps of use of the system embodiment.

FIG. 4E is a cross sectional view of a further embodiment of the medicament delivery system.

FIG. 4F-4I are cross sectional views illustrating the steps of use of the embodiment of the medicament delivery system provided in FIG. 4E.

FIGS. 5A-5C are perspective views of a further embodiment of the medicament delivery system and illustrate the steps of use of the system.

FIGS. 5D-5F are cross sectional views of the embodiment of the medicament delivery system steps provided in FIGS. 5A-5C.

FIG. 6A is a perspective view of another embodiment of the medicament delivery system.

FIGS. 6B-6C are partial views of the medicament delivery system embodiment shown in FIG. 6A.

FIG. 7A is a perspective view of an embodiment of the medicament delivery system.

FIGS. 7B-7C are cross sectional views illustrating steps of use of the embodiment of the medicament delivery system provided in FIG. 7A.

FIG. 8A is a cross sectional view of another embodiment of the medicament delivery system.

FIG. 8B is a front elevational view of the cross sectional view of the medicament delivery system shown in FIG. 8A.

FIG. 9 is a cross sectional view of an embodiment of a medicament delivery system including a manual actuation mechanism.

FIG. 10 is a cross sectional view of a further embodiment of an actuation mechanism of the medicament delivery system.

FIGS. 11A-D are cross sectional views illustrating steps of the use of the embodiment of the system provided in FIG. 10.

FIG. 12A is a rear perspective view of an embodiment of a reusable housing for receiving a container.

FIG. 12B is a cross sectional view of the embodiment of FIG. 12A for a reusable housing taken along line D-D in FIG. 12A.

FIG. 13A is a perspective view of a reusable housing embodiment for receiving a container.

FIG. 13B is a sectional view of the lower portion of the reusable housing embodiment shown in FIG. 13A being held by a user.

FIG. 13C is a perspective view of the reusable housing embodiment shown in FIG. 13A being held by a user.

FIG. 14A is a plan view of a reusable housing embodiment for receiving a container.

FIG. 14B is a elevational view of the reusable housing embodiment shown in FIG. 14A.

FIG. 14C is a front side perspective view of the reusable housing embodiment of FIG. 14A.

FIG. 15A is a front side elevational view of another reusable housing embodiment for receiving a container and a container embodiment.

FIG. 15B is a back side elevational view of the reusable housing embodiment of FIG. 15A.

FIG. 15C is a perspective view of the reusable housing embodiment of FIG. 15A during use.

FIGS. 16A-C are container embodiments comprising varying contaminant barriers.

FIG. 17A is a cross sectional view of a container embodiment alternative to FIG. 16B.

FIGS. 17B-C include an exploded view of the container embodiment of FIG. 17A.

FIG. 18 is a flow chart demonstrating an example of a method of manufacturing the container embodiment of FIG. 17A.

FIG. 19A is a perspective view of an embodiment of a container partially surrounded by a cover portion.

FIG. 19B is an elevational view of the container embodiment of FIG. 19A.

FIG. 19C is a cross sectional view of the container embodiment taken along line Y-Y of FIG. 19B.

FIG. 19D is an elevational view of the container embodiment of FIG. 19B with the cover portion removed and disposed below the container.

FIG. 19E is a cross sectional view of the container embodiment and cover portion taken along line Z-Z of FIG. 19D.

FIG. 20A is a perspective view of a container embodiment including a cover portion.

FIG. 20B is an elevational view of the container embodiment of FIG. 20A.

FIG. 20C is a cross sectional view of the container embodiment taken along line A-A of FIG. 20B.

FIG. 20D is a side perspective view of the container embodiment of FIG. 20A with the cover removed.

FIG. 20E is an elevational view of a cover portion.

FIG. 20F is a cross sectional view taken along line B-B of FIG. 20E.

FIG. 21A is a front perspective view of a container embodiment.

FIG. 21B is a front perspective view from below of the container embodiment shown in FIG. 21A.

FIG. 21C is a side elevational view of the container embodiment shown in FIGS. 21A, 21B.

FIG. 21D is a cross sectional view taken along line C-C of FIG. 21C.

FIG. 22A is a perspective view of a shell portion with a container held therein and a triggering mechanism.

FIG. 22B is a perspective view of the shell portion embodiment of FIG. 22A with the container embodiment removed from the shell portion.

FIG. 23A is a front elevational view of the shell portion embodiment and container of FIG. 22A.

FIG. 23B is a cross sectional view of the shell portion embodiment and container taken along line E-E of FIG. 23A.

FIG. 24A is a side elevational view of the shell portion embodiment and container of FIG. 23A.

FIG. 24B is a cross sectional view of the shell portion embodiment and container of FIG. 24A taken along line F-F.

FIG. 25A is a perspective view of a Smartphone reusable housing embodiment with a container embodiment received therein.

FIG. 25B is a perspective view of a Smartphone reusable housing embodiment with a container embodiment removed there from.

### DETAILED DESCRIPTION

A beneficial advance over the prior art would be to provide an injection cartridge or container and an injection training cartridge or container which can be used for a variety of different medications currently on the market and those not yet available. This container may be universal in nature such that it can advantageously fit within an injection device housing or injection training device housing, and may be inserted into and ejected from an injection device or injection training device currently available in the market or developed in the future. Furthermore, the container may contain any medicament that is delivered via injection to a user. The container may include safety measures to prevent the sticking of oneself with an injection member or needle when not in use, and may also include various means for maintaining sterility of the components of the container as well as any medicament housed there within.

In non-limiting embodiments of the system and container, a unidirectional force may be applied through a stopper of the container to effect a two step process, for example, to deliver the injection member from the housing into a target area of a user, and deliver medicament from the container through the injection member into the target area of a user. Thus, the application of a unidirectional force is able to achieve two different outcomes: insertion of injection member in one step and ejection of medication in a second step. This elegant arrangement also decreases the complexity of the structural components involved, as well as the manufacturing processes involved.

In non-limiting embodiments described herein, the order of movement of the first housing relative to the second housing of the container and the movement of the stopper relative to the first housing occurs by following the path of least resistance first, followed by the path of greater resistance. Consequently, the injection member will be delivered from the second housing into the target area of the user before the medicament is delivered from the first housing of the container, as less resistance occurs when the first housing moves relative to the second housing and the injection member pierces the skin of the user, than when the stopper moves in a first direction relative to the first housing delivering medicament through the injection member into the target area of the user.

Furthermore, for enhancing the user experience during an injection, it is beneficial to insert the injection member into the skin of the user quickly, and deliver the medicament from the container through the injection member to the user more slowly. A great deal of pain is associated with the delivery of the medicament through the injection member into the user, and consequently, slowing this process down decreases the discomfort to the user. In contrast, the more quickly the needle is injected into the user, the less anxiety build-up the user will have before and during an injection, and the user will experience less pain associated with the injection. Consequently, in some, non-limiting embodiments that will be described in greater detail below, different actuation mechanisms are used to actuate the container and system described herein, such that the delivery of the injection member from the container occurs more quickly, and the delivery of medicament from the container, through the injection member and into the user occurs more slowly.

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to those skilled in the art to which the invention pertains.

It is to be noted that the terms “first,” “second,” and the like as used herein do not denote any order, quantity, or importance, but rather are used to distinguish one element from another. The terms “a” and “an” do not denote a limitation of quantity, but rather denote the presence of at least one of the referenced item. The modifier “about” used in connection with a quantity is inclusive of the stated value and has the meaning dictated by the context. It is to be noted that all ranges disclosed within this specification are inclusive and are independently combinable. Furthermore, to the extent that the terms “including,” “includes,” “having,” “has,” “with,” or variants thereof are used in either the detailed description and/or the claims, such terms are intended to be inclusive in a manner similar to the term “comprising.”

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. As a non-limiting example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 7.

### DEFINITIONS

A “solenoid” as described herein may include any type of solenoid known in the art. In one embodiment, the solenoid may be a simple solenoid with only two stages or phases, either on or off, in or out, for example. In another embodiment, the solenoid may be a proportional solenoid which can be used to convert an electric control signal into a proportional mechanical force. In contrast to the on-off simple solenoid where only the start and end positions are relevant, all intermediate stages of the proportional mechanical force of the proportional solenoid movement are relevant. With a proportional solenoid, the speed with which the solenoid moves from one position to the next can be controlled. Non-limiting examples of proportional solenoids are provided in U.S. Pat. No. 7,069,951 and U.S. Pat. No. 4,651,118.

A proportional solenoid may be particularly beneficial in controlling the speed with which a medicament is delivered from the medicament delivery system as disclosed herein. Proportional solenoids are often used in applications requiring variable and precise control. The mechanism allows the ability to achieve varying position of the solenoid and/or force provided by the solenoid. In certain instances, it may be beneficial to speed up the movement wherein, in a non-limiting example, a first housing moves into a second housing of the system and an injection member is ejected from the second housing, and to slow down the movement of a stopper in a first direction relative to the first housing to dispel the medicament contained therein into a target area of a user. Therefore, in a two step process one step can be completed faster than the other step by using both a simple solenoid and a proportional solenoid or with only one single proportional solenoid, in non limiting examples.

Various actuation mechanisms are discussed and described herein. The term “actuation mechanism” as used herein may refer to a mechanism used to actuate movement of one part of a device relative to another part of a device. For example, the actuation mechanism may be configured to move a stopper of a container in a first direction relative to a first housing of the container. There may be one or more actuation mechanisms in a device or system as described herein. Furthermore, in non-limiting examples, the actuation mechanism may include a shaft portion associated with the stopper, in one embodiment, pressurized air, an electrical signal, or any other means of actuation known to those of skill in the art. Other mechanical and structural-type actuation mechanisms are also described herein including a first housing displacement component and a stopper displacement component which include projection members in some non-limiting embodiments, wherein the projection members interact with the various components of the first housing and/or the stopper, for example, to displace the components within the container. Actuation mechanisms may include actuation members.

The term “associated” or “association”, as used herein, includes but is not limited to direct and indirect attachment, adjacent to, in contact with, partially or fully attached to, and/or in close proximity therewith. The term “in conjunction with” as used herein includes but is not limited to synchronously or near synchronous timing, the phrase may also include the timing of outputs, where one output directly follows another output.

The term “biased state” as used herein in reference to a spring may include either compression or extension of the spring. The term “contaminant barrier” as used herein includes a contact between two components of the container herein, such as, for example, the first and second housing such that it prevents contaminants from entering. A contaminant barrier may further be defined as a piece of material (o-ring, silicone gel, for example), a bearing providing contact between the first and second housings that seals the second housing, for example, or a seal between two components of the container such as the first and second housings. The first contaminant barrier, in one embodiment, may include a membrane which maintains the sterility of the injection member until the injection member pierces through the first contaminant barrier.

The term “injection member” as used herein includes a needle or other member used to traverse the skin of a user and inject fluid there through, in one example. Injection member may also include an injection simulation member which may include a blunt end probe or other similar object known in the art provided to mimic the sound, look, and/or feel of the injection by an injection member (i.e., a needle) in a training/simulation session without puncturing the skin of the user, such as a retractable structure or other such needle simulation structure, for example. One skilled in the art would realize that the injection simulation member can be made of any materials known in the art to provide the flexibility, and tensile modulus while maintaining the rigidity and stability to provide a simulated sensation of an injection without traversing the skin of a user, such as, a monofilament injection simulation member made form a resin with high elastic memory in a non-limiting example, which will bend and thereafter return to its original shape once it is removed from contact with the user.

The terms “medicament delivery system housing” and “reusable housing” may be used interchangeably herein. The medicament delivery system housing and reusable housing may be used with a medicament-containing container or a non-medicament containing container. The medicament delivery system housing and reusable housing described herein may be used to deliver medicament to a user and/or to train a user to deliver medicament. Consequently, the housings may be used with containers containing medicament in some embodiments in which the housing and container is being used to deliver medicament to a user and in addition, in some non-limiting embodiments, to train a user to deliver medicament or to walk a user step by step through medicament delivery. In an alternative embodiment, the housing may be used with a container having no medicament to train a user to use a medicament delivery device. In some non-limiting embodiments, the housing may be reusable, in other non-limiting embodiments, the housing may be disposable.

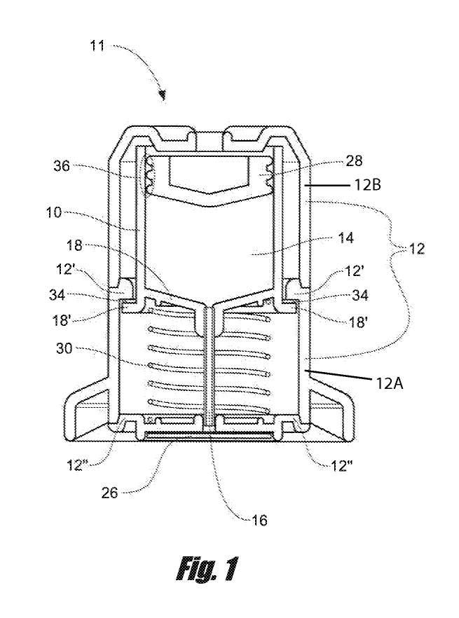

In the cross sectional view of an embodiment of a container 11 in FIG. 1, a first housing 10 and a second housing 12 are provided, wherein the first housing 10 includes a medicament 14. A first contaminant barrier 26 is disposed at a lower portion 22 of the second housing 12 so as to prevent any contaminants from entering the second housing 12 or contacting the injection member 16 provided there within. A second contaminant barrier 34 can be provided between a lower portion of the first housing 18 and may be disposed between a first housing flange 18′ and the second housing 12, specifically a second housing upper flange 12′ as shown in FIG. 1 to prevent contaminants from entering the second housing 12 and/or contacting the injection member 16. The first housing flange 18′ moves between the second housing upper flange 12′ and the second housing lower flange 12″ in one embodiment.

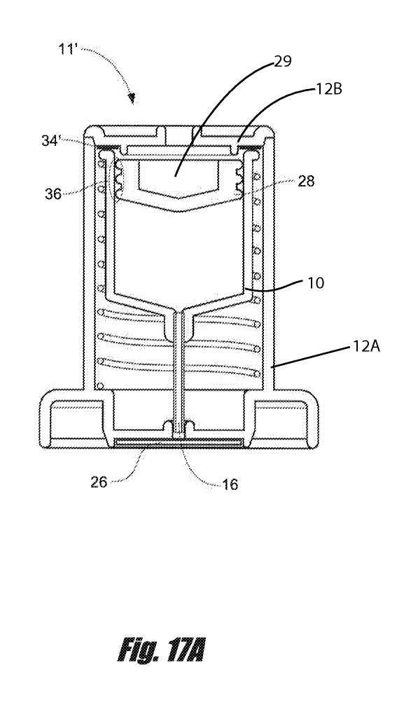

A third contaminant barrier 36 is provided between a stopper 28 and the first housing 10 to prevent contaminants from entering the first housing 10 and/or coming in contact with the medicament 14 housed therein. A spring 30 is disposed between a lower portion 18 of the first housing and a lower portion 22 of the second housing. In an embodiment, actuation of the container 11 allows the first housing 10 to move relative to the second housing 12 in a first direction such that the injection member 16 traverses the first contaminant barrier 26, and the spring 30 is in a biased state. Thereafter, the stopper 28 moves in a first direction relative to the first housing 10 to dispel any medicament 14 contained within the first housing 10. The medicament 14 is dispelled through the injection member 16. The first housing 10 then moves relative to the second housing 12 in a second direction, releasing the spring 30, and the injection member 16 is withdrawn into the second housing 12 to prevent an unintentional contact with the injection member 15. The second housing 12 may surround, in one embodiment, the container 11 as shown in FIG. 1. The second housing 12 is shown as two pieces, 12A and 12B in FIG. 1, but may also be one continuous component 12 as shown in other Figures herein (FIG. 17A, for example). The second housing flanges 12′, 12″ may also be continuous with the rest of the second housing 12, for example.

Various actuation devices and mechanisms for actuating the container 11 will be discussed in more detail herein. In some embodiments, the first and/or second housings 10, 12 may include a transparent material such that a user can see through the first and/or second housing 10, 12 into the container 11. In a particular non-limiting embodiment, a user of the container 11 may be able to view the medicament 14 through the transparent first housing 10 and through a transparent portion of the second housing 12 such that a user may view an amount of medicament 14 that remains in the container 11, however the portion of the second housing 12 surrounding the injection member 16 may not be transparent so that the injection member 16 is hidden from the view of the user.

While the embodiments described and provided in the Figures herein include a spring disposed between the first and second housings used to move the first housing 10 relative to the second housing 12 in a second direction, and to retract the injection member 16 within the second housing 12 following an injection, the spring 30 is not required for the retraction of the injection member 16 and the first housing 10. It is contemplated herein that a number of different variations of retraction mechanisms can be used. For example, a tension spring can be associated with the second housing and a portion of the first housing, wherein the spring could be used to move the first housing relative to the second housing in a second direction to retract the injection member 16. Another embodiment may include an actuation mechanism which attaches onto or associates with the stopper or the first housing so as to retract the first housing, for example. In still further non-limiting embodiments, the second housing 12 may be forced downward over the injection member 16 by mechanisms know to one skilled in the art so as to envelop the injection member 16 to prevent unwanted sticks with the injection member 16.

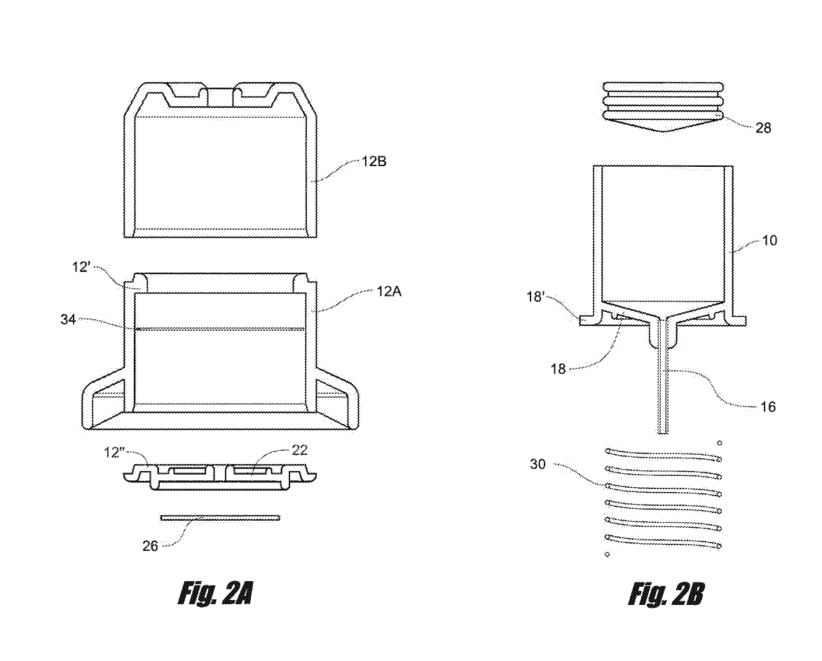

FIGS. 2A-B are an exploded view of a non-limiting embodiment of a container 11, wherein in FIG. 2A the second housing 12 is shown as two parts, a lower second housing portion 12A and an upper second housing portion 12B which are fused together to form the second housing 12. The lower portion of the second housing 22 is shown separate from the second housing 12A, 12B, but is also fused to the second housing 12A in operation. The first contaminant barrier 26 is provided as well as the second contaminant barriers 34 and 34′. FIG. 2B provides an exploded view of the other portions of the container 11 including the stopper 28, the first housing 10, having a lower portion 18 of the first housing and an injection member 16 associated with the lower portion 18 of the first housing. A cross sectional view of the spring 30 is also provided in FIG. 2B.

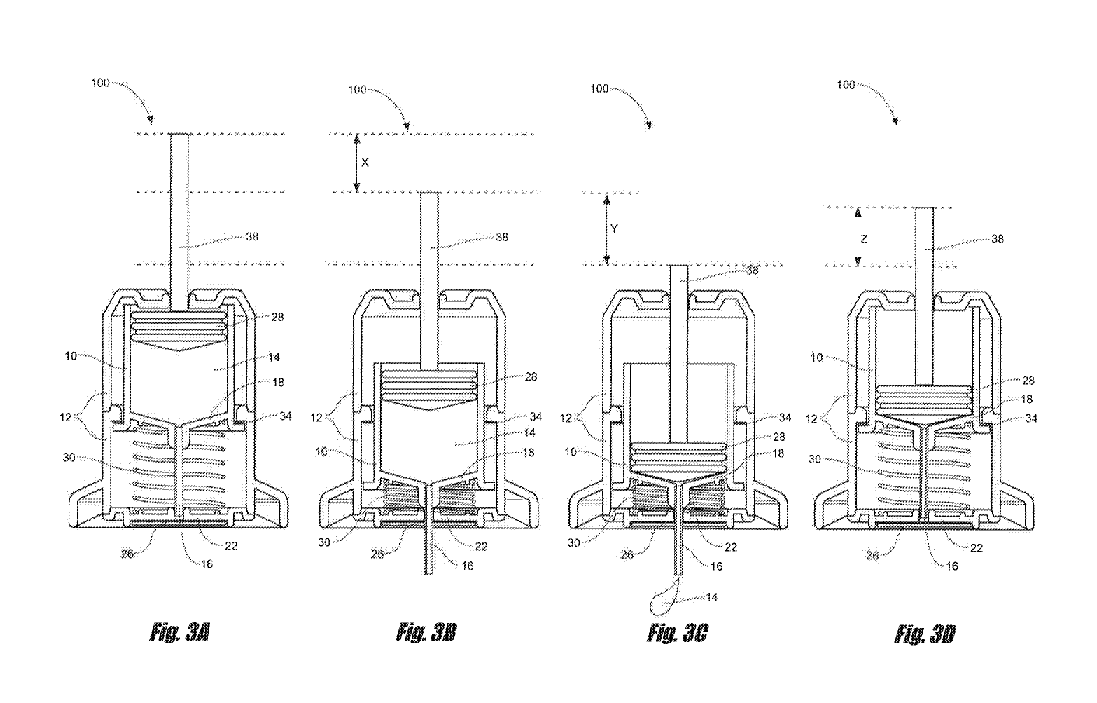

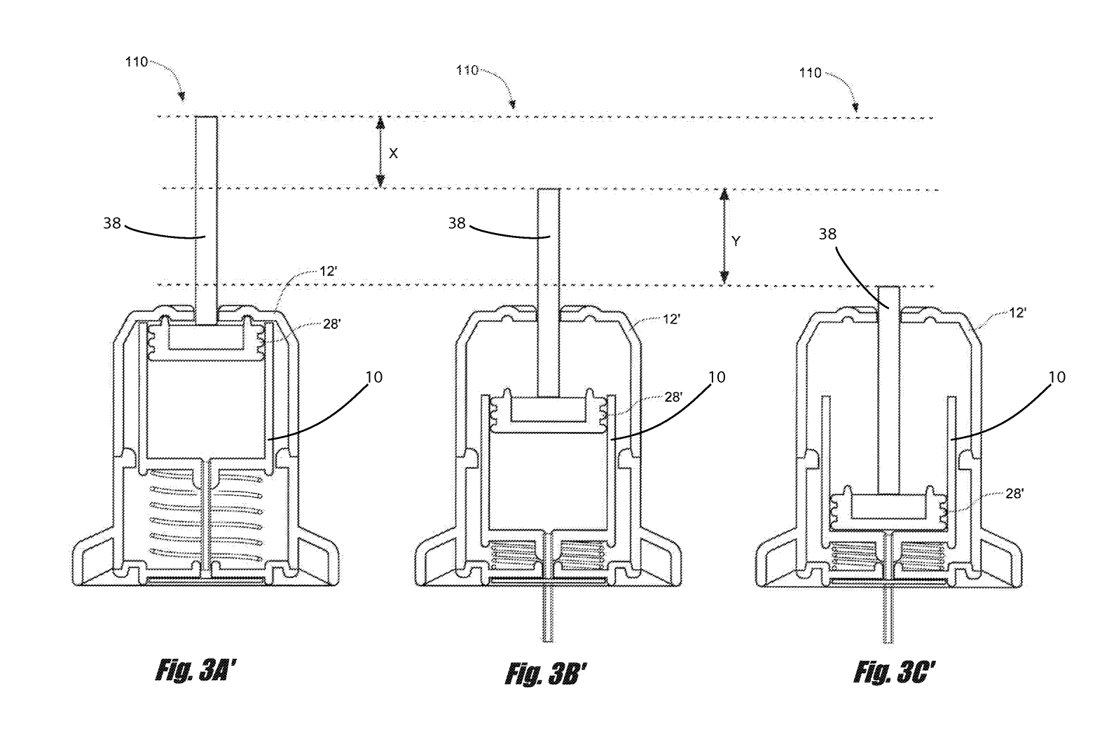

FIGS. 3A-3D are cross sectional views of an embodiment of the medicament delivery system 100 illustrating steps of the use of the system according to one embodiment. In the embodiment of FIGS. 3A-3C an actuation mechanism 38 interacts with the stopper 28, wherein the first housing 10 moves in a first direction relative to the second housing 12 as can be seen in FIG. 3B. This causes the injection member 16, associated with a lower portion 18 of the first housing 10 to traverse the first contaminant barrier 26 positioned at the lower portion 22 of the second housing 12, and the spring 30 to be in a biased state. Thereafter, the stopper 28 moves relative to the first housing 10 in a first direction as shown in FIG. 3C to deliver medicament 14 stored in the first housing 10 through the injection member 16.

In an embodiment, displacement of the actuation mechanism 38 is shown as X demonstrating the displacement of the actuation mechanism 38 during movement of the first housing 10 in a first direction relative to the second housing 12 such that the injection member 16 traverses the first contaminant barrier 26. This movement X provided between FIG. 3A and FIG. 3B can be rapid and no control over the displacement is required. It is preferable to inject an injection member (i.e., needle, in one embodiment) into a patient quickly, as aforementioned, in order to manage or decrease the pain associated therewith and lessen any anxiety a patient may develop regarding the injection. However, in contrast, delivery of medicament into a patient should be slow and controlled to lessen any pain associated therewith. Therefore, in one embodiment, the displacement of the actuation mechanism 38, shown as Y demonstrating the displacement of the actuation mechanism 38 during movement of the stopper 28 in a first direction relative to the first housing 10 between FIGS. 3B and 3C. This movement can be slow and controlled such that any pain associated with delivery of the medicament 14 from within the first housing 10 through the injection member 16 and into the patient can be slow and controlled in order to decrease any pain associated therewith.

Various actuation mechanisms can be used to actuate the container 11 as described herein; however, in order to effect a slow and controlled movement, a proportional solenoid or a motorized actuation mechanism can be used, in non-limiting examples, as shown in the Figures below. Any other speed or force-controllable mechanism as known in the art may also be used herein. FIG. 3D demonstrates the retraction of the injection member 16 into the second housing 12 to prevent unintended contact with the injection member 16 following an injection. This displacement can be accomplished by releasing the spring 30 so that the first housing 10 moves in a second direction relative to the second housing 12, thus retracting the injection member 16 into the second housing 12 in one example. Other methods of retracting the injection member 16 and moving the first housing 10 in a second direction relative to the second housing 12 are also discussed herein. The displacement of the actuation mechanism 38 once the injection is complete and the injection member 16 is retracted into the second housing 12, is denoted as Z. The stopper 28 may remain in the lower portion of the first housing 10 once the injection is complete.

FIGS. 3A′-3C′ provide a cross sectional view of an illustration of the steps of use of another embodiment of the medicament delivery system 110, wherein said system differs from the system 100 illustrated in FIGS. 3A-3C in that the second housing 12′ includes a different shape at its upper portion, wherein it is shaped to accommodate a stopper 28′ shaped so as to provide a seal between the stopper 28′ and the second housing 12′ without the need for a second contaminant barrier 34 as provided in the embodiment shown in FIGS. 1-3C. As described above in regard to FIGS. 3A-3C, the displacement X of the actuation member 38 as the first housing 10 moves relative to the second housing 12′ in a first direction and the displacement Y of the actuation mechanism 38 as the stopper 28′ moves relative to the second housing 12′ in a first direction to dispel medicament from the first housing 10 is illustrated herein. The system embodiment 110 will subsequently retract similar to the way in which the system embodiment 100 illustrated in FIGS. 3A-3C retracts following an injection.

The order of movement of the first housing 10 and the stopper 28 can be dictated by resistance as there is a greater level of resistance in delivering the medicament 14 through the injection member 16 and therefore moving the stopper 28 relative to the first housing 10 in a first direction, than there is in moving the first housing 10 relative to the second housing 12 to eject the injection member 16 through the first contaminant barrier 26, in some embodiments. Consequently, with only one actuation mechanism 38 acting on the stopper 28, the first housing 10 can move in a first direction relative to the second housing 12 before the stopper 28 moves in a first direction relative to the first housing 10, in an embodiment.

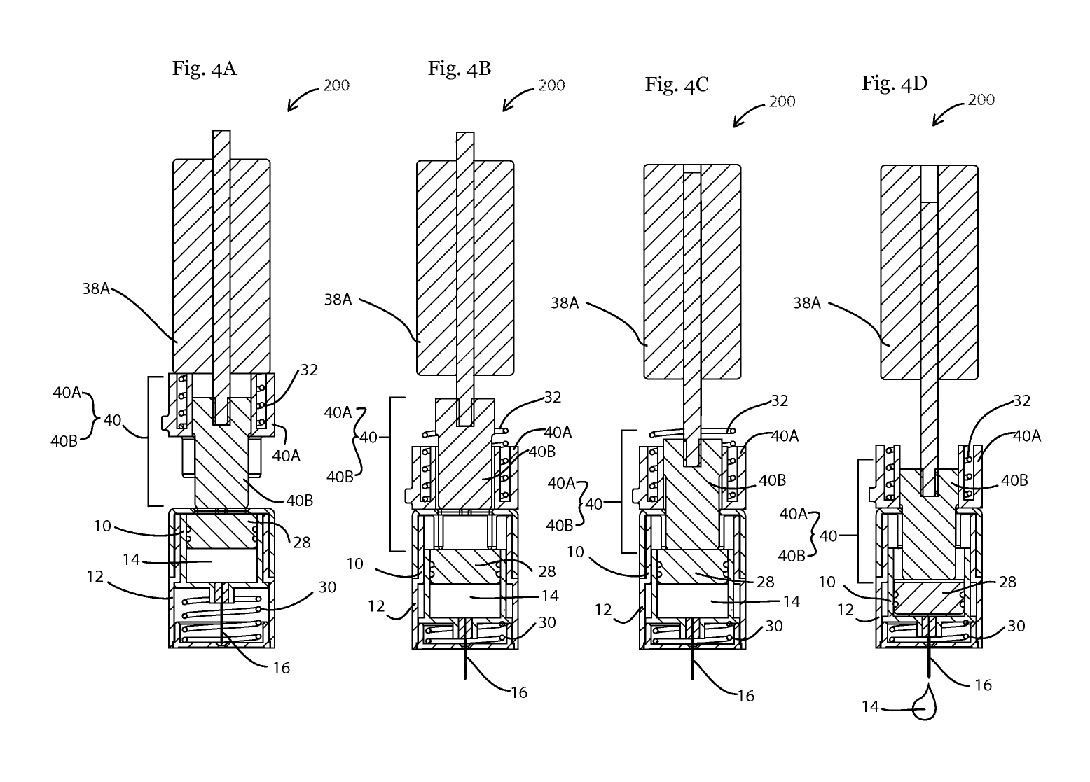

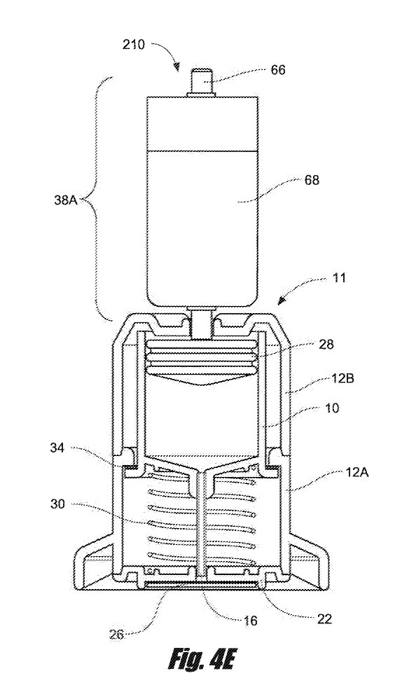

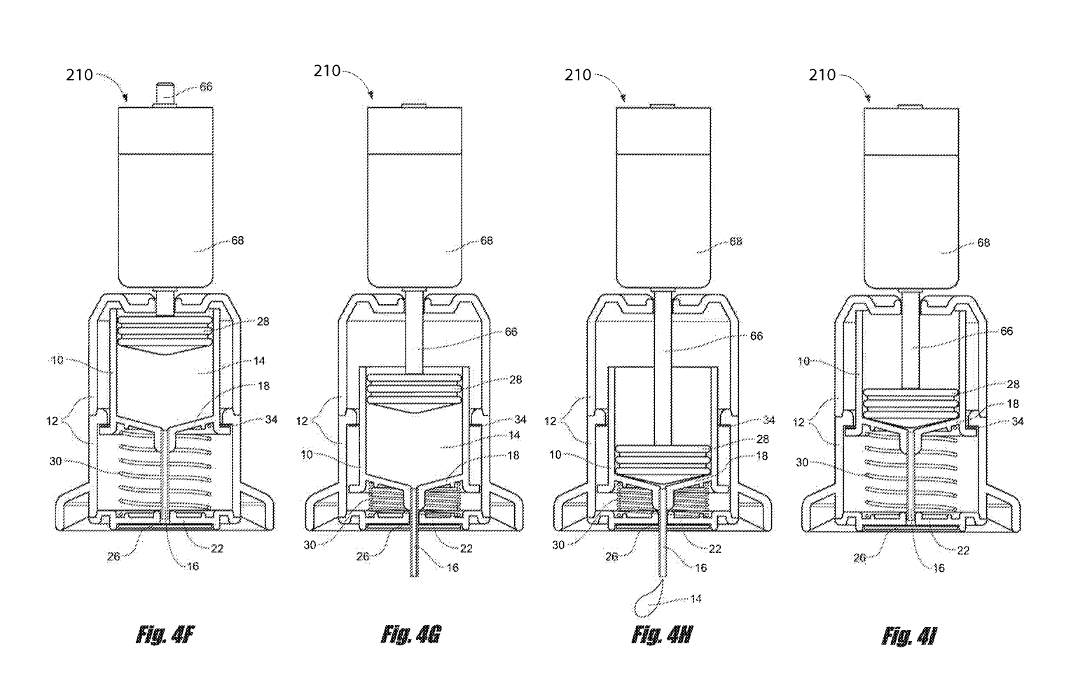

FIGS. 4A-D provide cross sectional views of a further embodiment of a medicament delivery system 200, wherein a displacement member 40 including a first housing displacement component 40A and a stopper displacement component 40B are provided to interact with the container 11 so as to activate the container 11. A first actuation mechanism 38A is also provided in the embodiment of the system 200, which interacts with the stopper displacement component 40B, the stopper displacement component 40B interacts with the stopper 28 so as to move the stopper 28 in a first direction relative to the first housing 10. In the embodiment of the system 200, the first housing displacement component 40A interacts with a portion of the first housing 10 to move the first housing 10 in a first direction relative to the second housing 12 to eject the injection member 16. This step typically occurs before the stopper 28 is moved relative to the first housing 10 in a first direction. A second spring 32 may be used, in non-limiting embodiments, to provide a force to move the first housing displacement component 40A relative to the first housing 10 in a first direction to move the first housing 10 in a first direction relative to the second housing 12, in one embodiment. In a non-limiting embodiment, the first actuation mechanism 38A is a solenoid as is shown in FIGS. 4A-D. In an alternative embodiment, the second spring 32 may not be used and the first actuation mechanism 38A may be used to move both the first housing displacement component 40A and the stopper displacement component 40B.

In an embodiment, the stopper displacement component 40B may include a catch or an engagement member to retract the first housing into the second housing, i.e., move the first housing 10 in a second direction relative to the second housing 12 following an injection in contrast to the spring 30, as already described herein. Alternatively, as described above, both the ejection and the retraction motions of the injection member 16 can be accomplished with the actuation mechanism 38A, for example, wherein a spring 30 is not required. The actuation mechanism 38A may be attached to the stopper 28 and/or the first housing 10 by any means currently known in the art, and therefore may provide movement of the stopper 28 and/or the first housing 10 in a first direction relative to the second housing 12 to eject the injection member 16 and deliver the medicament 14 through the injection member, as well as to move the first housing 10 and/or the stopper 28 in a second direction relative to the second housing 12 to retract the injection member 16 following an injection.

In a further embodiment, the second housing 12 may include one or a series of openings at its upper portion where the displacement member 40 contacts the second housing 12. In this embodiment, the displacement member 40 may include complementary projections to the openings in the second housing 12 to allow the displacement member 40 to contact the stopper 28 and the first housing 10 through the second housing 12 upper portion to actuate the container 11. These openings and projections may be provided as part of a safety feature to prevent unintended actuation of the container 11 when it is held in the hand of a user or when a user presses the upper portion of the second housing 12.

FIG. 4E provides a cross sectional view of a further embodiment of the medicament delivery system 210, wherein a proportional solenoid is provided as the actuation mechanism 38A. The proportional solenoid includes a plunger 66 and a coil housing 68, in a non-limiting embodiment. FIGS. 4F-4H provide a cross sectional view illustrating the steps of use of the embodiment of the medicament delivery system 210 provided in FIG. 4E, wherein the proportional solenoid is actuated in FIG. 4F, and in FIG. 4G, the first housing 10 is moved in a first direction relative to the second housing 12 so as to eject the injection member 16 through the first contaminant barrier 26. In FIG. 4H, the stopper 28 is moved relative to the first housing 10 in a first direction so as to deliver medicament 14 contained within the first housing 10 through the injection member 16 and into a user. FIG. 4I illustrates the retraction of the injection member 16 into the second housing 12 by release of spring 30 and movement of the first housing 10 in a second direction relative to the second housing 12.

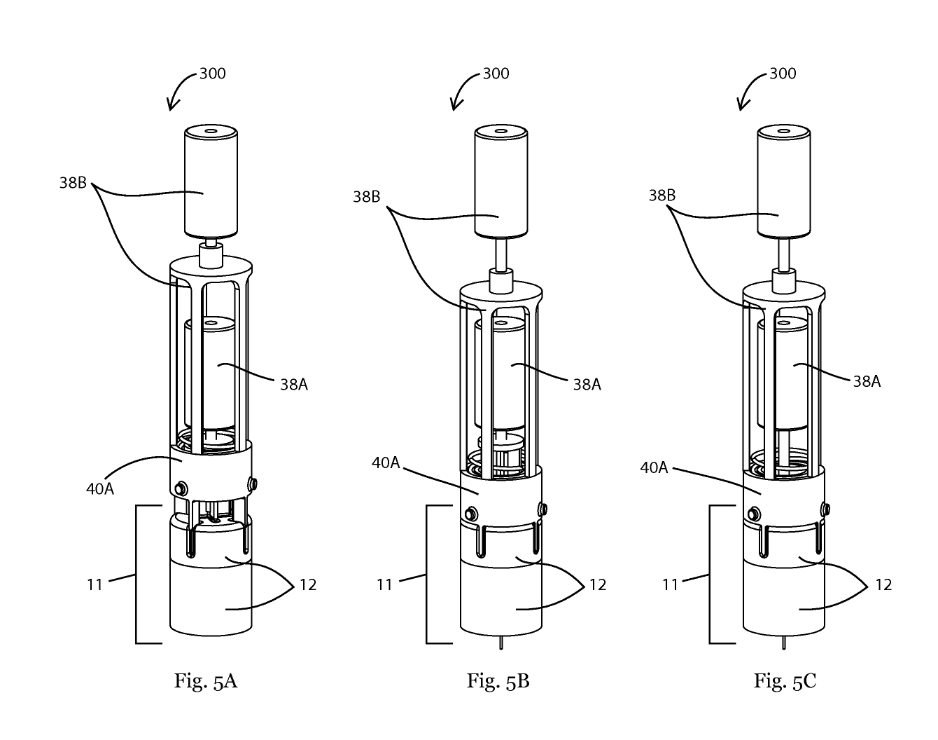

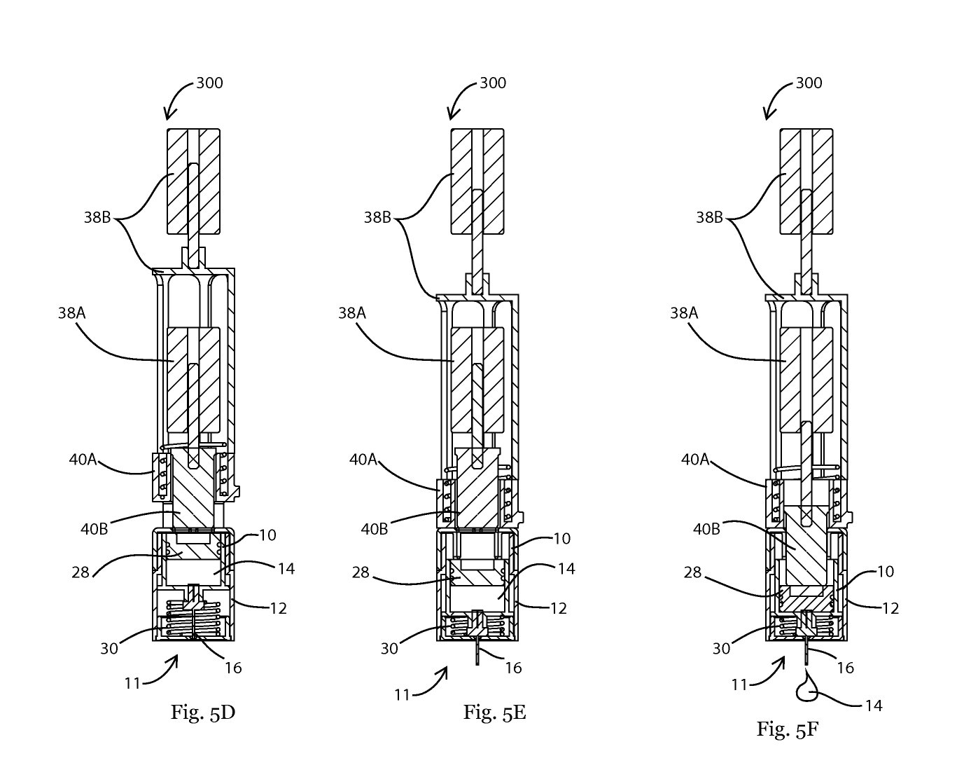

FIGS. 5A-C provide a perspective view of another embodiment of a medicament delivery system 300, and FIGS. 5D-F provide a cross sectional view of the embodiment of the system 300 shown in FIGS. 5A-C. The embodiment of the system 300 includes a first actuation mechanism 38A which interacts with and actuates the movement of a stopper displacement component 40B to displace the stopper 28 relative to the first housing 10, and a second actuation mechanism 38B which interacts with and actuates the movement of the first housing displacement component 40A to displace the first housing 10 relative to the second housing 12. In one embodiment, the second actuation mechanism 38B is activated before the first actuation mechanism 38A to displace the first housing 10 relative to the second housing 12 and deliver the injection member 16 there from, before the stopper 28 is displaced relative to the first housing 10 to deliver the medicament 14. As shown in FIGS. 5A-F, each of the first actuation mechanism 38A and the second actuation mechanism 38B may include a solenoid. In a particular non-limiting example, the embodiment 300 may include DC solenoids as first and second actuation mechanisms 38A, 38B as shown in FIGS. 5A-F. However, different types of actuation mechanisms known in the art including other actuation mechanisms described herein may also be used, which include but are not limited to ACME/lead screws, springs, compressed air, magnetic, flexinol/muscle wire, solenoids, standard DC motors, pancake motors, other motors, and any other related actuation mechanisms, in other non-limiting embodiments. The second actuation mechanism 38B is shown in FIGS. 5A-5F as interacting with the first housing displacement component 40A via a bracket structure. This bracket structure may vary and is not intended to be limiting of the invention described herein.

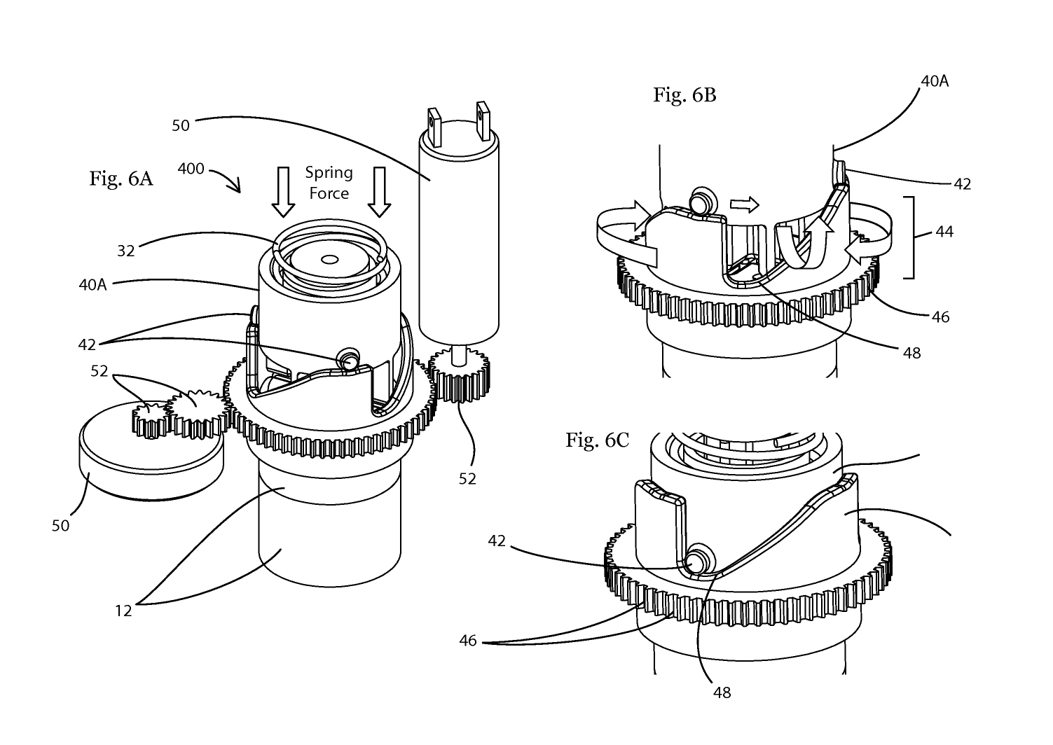

A further embodiment of the system 400 is provided in the perspective views of FIGS. 6A-C, wherein a barrel actuation mechanism 44 surrounds at least a portion of the container 11. The barrel actuation mechanism 44 is shown in FIGS. 6A-C as encircling at least a portion of the container 11. The barrel actuation mechanism 44 includes one or more grooves 48 and a series of barrel actuation mechanism nodules 46 on an outer surface of the barrel actuation mechanism 44. The barrel actuation mechanism nodules 46 are provided to interact with a motor 50 having motor nodules 52 which are complementary to the barrel actuation mechanism nodules 46. When the motor 50 is actuated, the motor nodules 52 interact with the barrel actuation mechanism nodules 46 to rotate the barrel actuation mechanism 44 around the container 11.

In one embodiment, as shown in FIGS. 6A-C, the barrel actuation mechanism 44 can be rotated in a counter clockwise direction around the container 11 and a second spring 32 in association with the first housing displacement component 40A is compressed such that projections 42 on the outer portion of the first housing displacement component 40A interact with the groove(s) 48 of the barrel actuation mechanism 44. The combination of the force provided by the second spring 32 on the first housing displacement component 40A and the grooves 48 of the barrel actuation mechanism 44 allow the first housing displacement component 40A to contact the first housing 10 of the container 11 to move the first housing 10 in a first direction relative to the second housing 12 and eject the injection member 16 from the second housing 12. The shape of the grooves 48 also serve to move the first housing displacement component 40A in a second direction relative to the container 11, so as to re-set the actuation mechanism as the barrel actuation mechanism 44 continues its rotation around the container 11. FIG. 6A shows two different motors 50 which can be used to rotate the barrel actuation mechanism 44. These are two variations of different motor orientations, one vertical DC motor and a pancake motor, are provided as examples. Only one motor 50 is needed to move the barrel actuation mechanism 44, and any additional types of motors known in the art may also be used to rotate the barrel actuation mechanism 44 as the examples provided herein are intended as non-limiting examples.

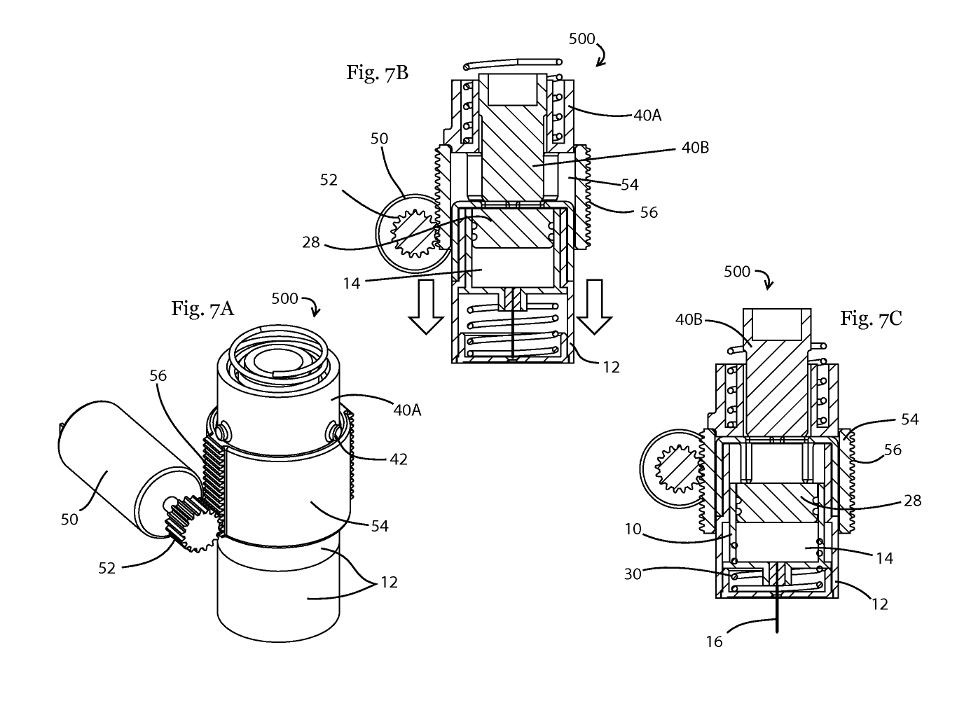

In a further embodiment of the medicament delivery system 500, a sleeve 54 including one or more sleeve nodules 56 surrounds at least a portion of the container 11. The sleeve 54 in FIG. 7 is shown as encircling the container 11. The nodules 56 on the sleeve interact with the motor nodules 52 such that when the motor 50 is activated, the motor nodules 52 cause the sleeve 54 to move in a first direction relative to the container 11, wherein the sleeve 54 is attached to a portion of the first housing displacement component 40A to move the first housing displacement component 40A in a first direction relative to the container 11, wherein the first housing displacement component 40A interacts with the first housing 10 to move the first housing 10 in a first direction relative to the second housing 12 to eject the injection member 16 there from. Thereafter, the stopper displacement component 40B can be moved in a first direction to move the stopper 28 relative to the first housing 10 in a first direction such that any medicament 14 stored within the first housing 10 is delivered through the injection member 16 into a patient target site. Movement of the stopper displacement component 40B can be accomplished by any means already described herein, or in an alternative embodiment, a series of nodules may be provided on an outer surface of the stopper displacement component 40B and an additional motor 50 including motor nodules 52 can interact with the stopper displacement component nodules to move the stopper displacement component in a first direction relative to the container to effect movement of the stopper 28 relative to the first housing 10. A spring force can provide movement of the first housing displacement component in a non-limiting embodiment, as in FIGS. 6A-C. In another non-limiting embodiment, the motor 50, motor nodules 52 and sleeve nodules 56 provides movement of the first housing displacement component 40A.

This embodiment including the sleeve 54 and sleeve nodules 56 in association with the motor nodules 52 can also be used to move the sleeve 54 in a second direction relative to the container 11 as desired, allowing the container 11 components to re-set and the first housing 10 and the stopper 28 to move in a second direction relative to the second housing 12 and the first housing 10, respectively. This can assist in retracting the injection member 16 back into the container 11 to prevent unwanted contact with the injection member 16 following use of the container 11 (i.e., following an injection). In a non-limiting embodiment, the spring 30 may not be needed wherein the sleeve nodules 56 and motor nodules 52 are used to allow the container components to reset as described herein such that the injection member 16 is retracted back into the container 11.

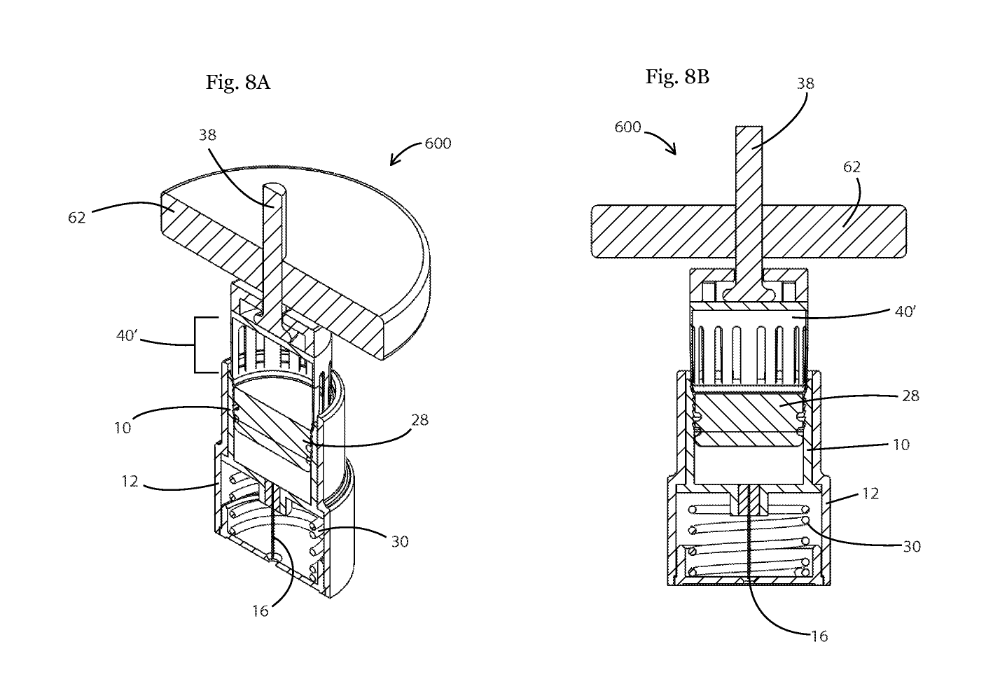

In yet a further medicament delivery system embodiment 600 shown in FIGS. 8A-8B, a pancake motor 50 is shown as associated with the actuation mechanism 38, wherein when the motor 50 is activated, it activates the actuation mechanism 38 to move the first housing 10 relative to the second housing 12 of the container 11 and to move the stopper 28 relative to the first housing 10 as described herein. The DC pancake motor 62 of FIGS. 8A-8B is shown as a non-limiting example of a variation of motor 50 that can be used to activate the system 600. FIG. 8 provides an alternative embodiment of a displacement member 40′ including a flexible member with or without fingerlike portions and openings disposed between the fingerlike portions (fingerlike portions and openings shown in FIG. 8A-B) wherein a downward pressure on the displacement member 40′ by the actuation mechanism 38 causes the fingerlike portions to force the first housing 10 to move in a first direction relative to the second housing 12, to eject the injection member 16. Thereafter, due to the flexibility of the flexible member, once the injection member 16 is ejected, the fingerlike portions shift inward such that they contact the stopper 28, and continued force onto the displacement member 40′ moves the stopper 28 in a first direction relative to the first housing 10 to deliver the medicament 14 within the first housing 10 through the injection member 16. The actuation mechanism 38 lifts the displacement member 40′ releasing the biased spring 30, such that the first housing 10 moves in a second direction relative to the second housing 12, and the injection member 16 is retracted into the second housing 12.

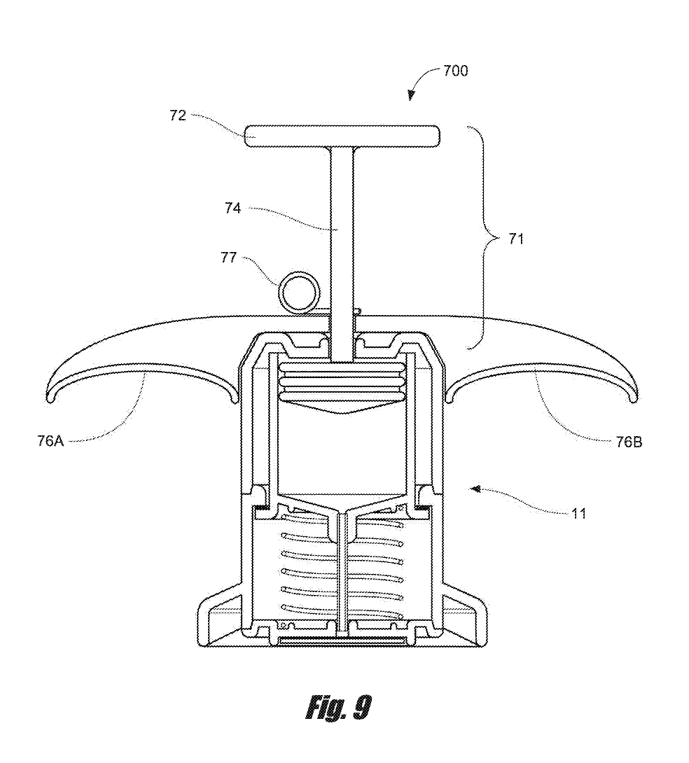

FIG. 9 provides a non-limiting embodiment of a manual actuation mechanism 71 used to activate the container 11. The manual actuation mechanism may include a thumb pad 72, a shaft 74, and finger grips 76A, 76B, in a non-limiting embodiment. Other configurations for the manual actuation mechanism 71 as known in the art are contemplated herein. A safety mechanism 77 may be provided in the embodiment of FIG. 9 so as to prevent an unintentional activation of the container 11. The safety mechanism 77 may act as a locking mechanism, in one embodiment, such that it must be removed prior to activating the manual actuation mechanism 71 to actuate the container 11. Other variations of locking mechanisms or safety mechanisms known in the art are contemplated herein. The safety mechanism 77 is not meant to be limiting, but only one example of a type of safety mechanism which can be used to prevent the unintended actuation of the manual actuation mechanism 71 of FIG. 9.

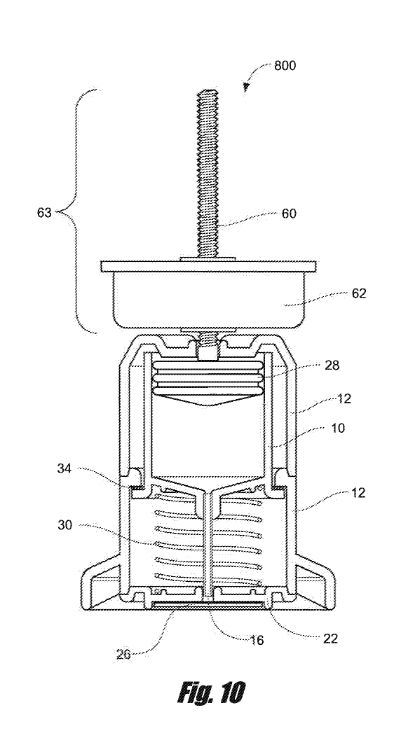

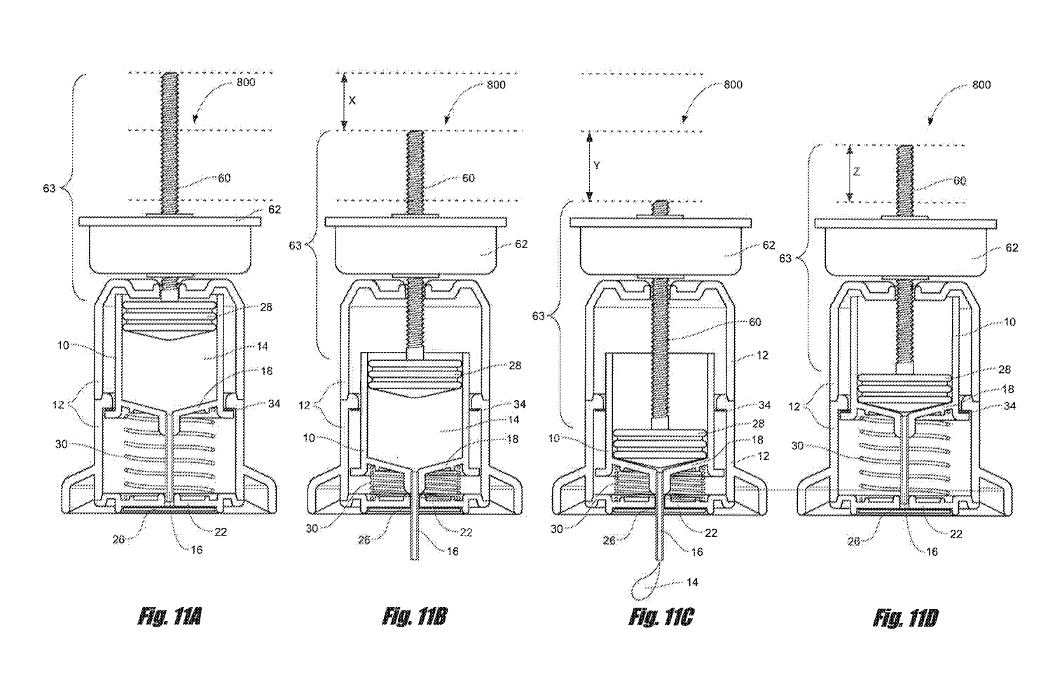

FIG. 10 includes a further embodiment of the medicament delivery system 800, wherein a linear servo actuation mechanism 63 is provided to activate the container 11. The linear servo actuation mechanism 63 includes a motor 62 (e.g. a DC motor) and a lead screw 60, in one embodiment. When the motor 62 is activated, the lead screw 60 is twisted down into the container 11 via the second housing, such that it contacts the stopper 28. The force of the lead screw 60 on the stopper causes the first housing 10 to move in a first direction relative to the second housing 12 to eject the injection member 16, before the stopper 28 is moved in a first direction relative to the first housing 10 to deliver medicament to the injection member, if medicament is contained within the first housing 10. Once the medicament has been delivered through the injection member 16, the spring 30 is released and the first housing 10 moves relative to the second housing 12 in a second direction such that the injection member 16 is retracted into the second housing 12 to prevent unintended contact with the injection member 16. The steps described in this embodiment are illustrated in FIGS. 11A-11D. As described herein, the retraction of the injection member 16 is not limited to the use of a spring 30 as shown in the drawings herein. Additional mechanisms discussed herein can be used to move the first housing 10 in a second direction relative to the second housing 12, and therefore retract the injection member 16, or furthermore, to solely retract the injection member 16 without the movement of the first housing 10 in a second direction relative to the second housing 12.

In a further embodiment, the medicament delivery system is provided wherein a sensor, for example, a contact sensor, is associated with the container, said contact sensor configured to detect contact between the sensor and a patient, wherein an output signal is provided to a processor based on the contact detected by the contact sensor. The contact sensor may be provided on a lower portion of the container (on a lower portion of the second housing or on the first contaminant barrier, for example) such that removal of the container from the user signals no contact signal to the processor. This function enables the system to detect if the container or the delivery system has been removed from the target injection site of the patient during an injection in order to prevent or minimize wet injections. Therefore, the processor may be associated with the stopper and a motor associated with either the first housing and/or the stopper, such that when the contact sensor detects no contact between the patient and the contact sensor, the processor stops the motor from moving the first housing in a first direction relative to the second housing and/or the stopper in a first direction relative to the first housing to prevent the medicament from being delivered through the injection member. In addition to, or in place of a sensor used to detect contact or lack thereof between the user and the container or system, other means to detect contact known in the art are contemplated herein including, but not limited to contact sensors or contact switches. Other types of sensors may be included, associated with the container or a housing described herein to provide guidance to a user when using the system, container, actuation mechanism(s) or housing(s) described herein.

Following use of the injection member for injection, in some embodiments herein, the injection member is retracted into the container such that the container can be safely removed from a reusable housing for disposal, or in some non-limiting embodiments the needle can be re-used in a subsequent injection. The retraction of the needle into the container prevents unwanted and accidental needle sticks by the patient during the manipulation of the container between its removal from the housing and disposal in a sharps container or during storage of the injection device until later use.

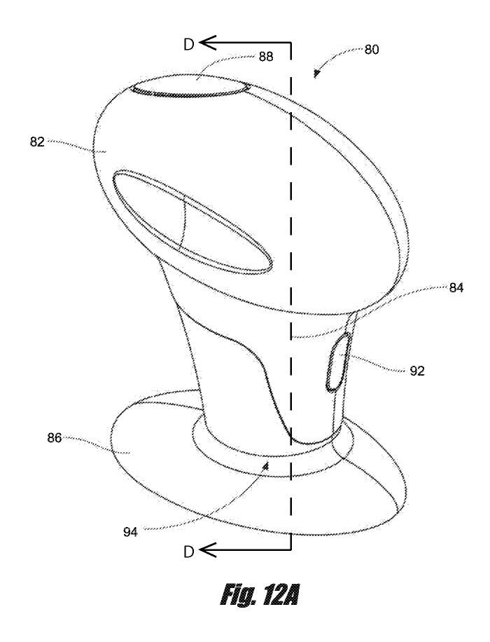

In one embodiment, a medicament delivery system housing 80 for use with a removable medicament container 11 is provided as illustrated in FIGS. 12A-12B. The housing 80 includes a generally ovoid shaped head portion 82 a shaft portion 84 including a top end 85 and a bottom end 87, the top end 85 is associated with the head portion 82. The housing 80 further includes a base 86 associated with the bottom end 87 of the shaft portion 84, the base 86 being wider than the shaft portion 84, a socket 94 (shown in the cross sectional view of FIG. 12B) configured to receive the container 11, The housing embodiment 80 further includes an actuation button 88 on said generally ovoid shaped head portion 82, an ejection mechanism 92 for releasing the container 11 when situated within the housing 80, and an actuation mechanism 89 associated with the actuation button 88, said actuation mechanism 89 configured to deliver medicament when the container 11 is received within the housing 80 and the actuation button 88 is activated. FIG. 12B provides a cross sectional view of the housing embodiment 80 of FIG. 12A, wherein a solenoid 98 is shown. The solenoid 68 activates the container 11 upon actuation. The solenoid 68 may include a coil housing and a plunger in one embodiment. A primary or secondary battery 69 may be included as the power source for the housing 80 to activate the solenoid 68 or any other member within the housing 80 requiring power. However, the power source is not limited to a battery or a particular type of battery. Any other power source as known in the art may be included to activate any of the components of the housing 80 or the container 11 herein. A safety switch or contact sensor 91 is provided at a lower portion of the housing 80, where the base 86 contacts a surface of the patient to be injected. The contact sensor or safety switch 91 will allow activation of the housing 80, and thus the container 11, when the safety switch or contact sensor 91 is in contact with a surface such as a target region for injection on a patient or user.

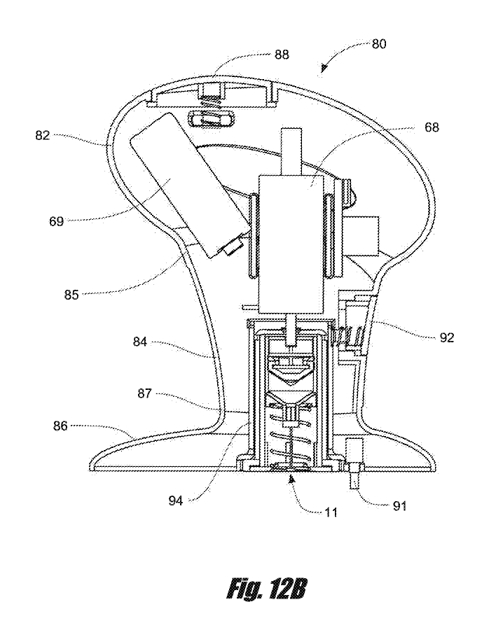

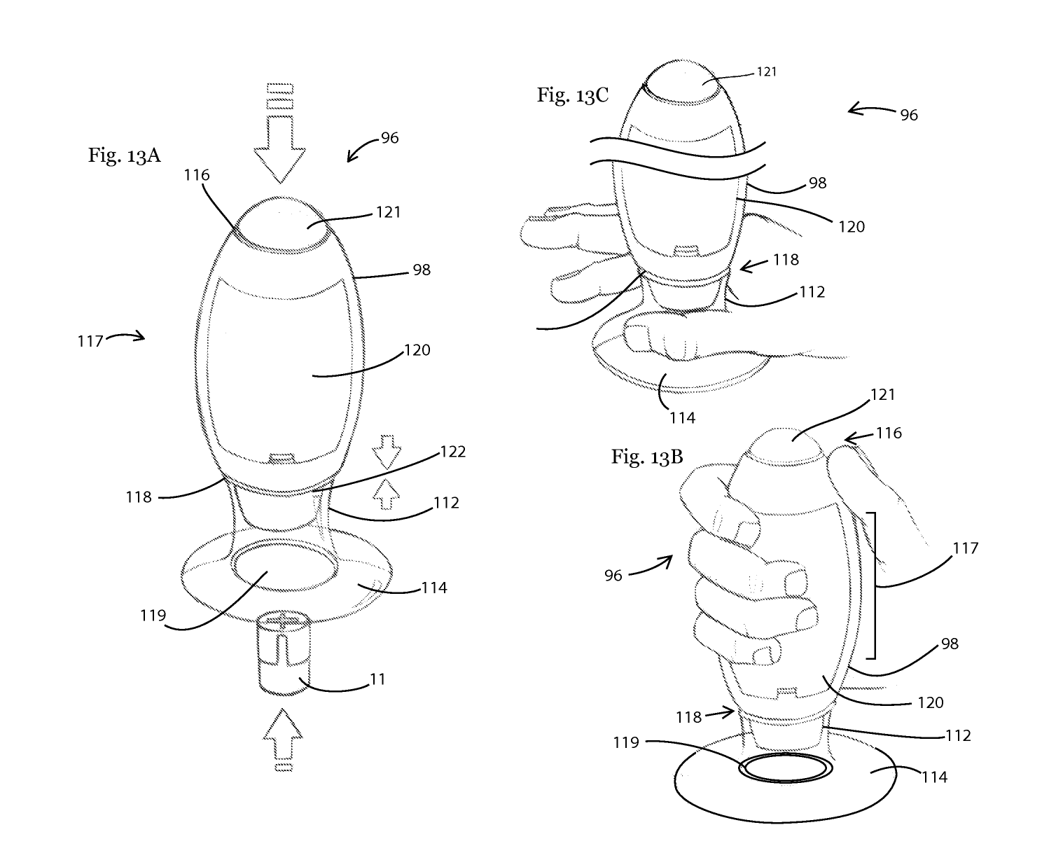

In a further embodiment, another medicament delivery system housing 96 for use with a removable medicament container 11 is provided as illustrated in the views of FIGS. 13A-C. The housing 96 includes a generally cylindrically shaped head portion 98 comprising a first end 116 and a second end 118 having a smaller diameter than a middle section 117 of the head portion 98. The housing 96 further includes a shaft portion 112 associated with the head portion 98, wherein in a resting position, an opening 122 is disposed between at least a portion of the shaft portion 112 and the head portion 98, a base 114 associated with the shaft portion 112, the base 114 provided for resting the housing 96 on a surface, and a socket 119 or opening in the base 114 configured to receive a container 11. The head portion 98 includes an actuation button 121 located at the first end 116 thereof, the shaft portion 112 comprises a compartment 120 therein for receiving and/or storing a power source. A locking mechanism 123 is provided and can be disposed within the head portion 98 of the housing 96, wherein said locking mechanism 123 is released when the shaft portion 112 and the head portion 98 are in contact with one another and the opening 122 between the shaft portion 112 and the head portion 98 is removed. The housing 96 further includes an actuation mechanism configured to activate the container 11 when the container 11 is received within the housing 96, the actuation button 121 is activated, and the locking mechanism 123 is released.

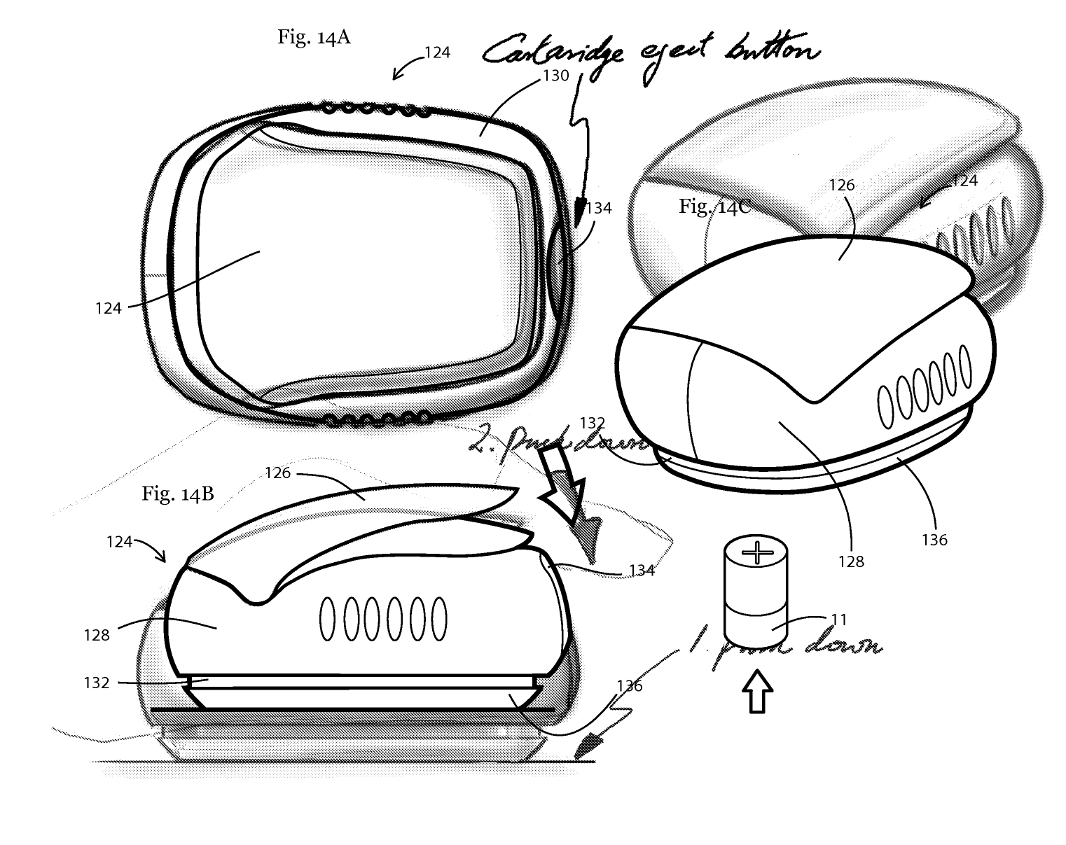

FIGS. 14A-C provide a top plan view (FIG. 14A), a side elevational view (FIG. 14B) and a front perspective view (FIG. 14C) of a medicament delivery system housing 124 for use with a removable medicament container embodiment 11. The housing 124 includes a generally oblong spherical shaped body 128, wherein the body 128 includes an upper surface 130 and a lower surface 132, the lower surface 132 configured for resting on a user. The lower surface 132 includes an opening 133 (not shown in the FIGS.) configured to receive a container 11. The upper surface 130 includes a deformable actuation mechanism component 126, the actuation mechanism component 126 is configured to activate the container 11 when the component 126 is deformed. The deformable actuation mechanism component 126 is configured for receiving a palm of a hand of a user, and can be deformed by pressing downwardly on the deformable actuation mechanism component 126 with the hand of the user when the housing 124 is resting on a surface, for example. Once the container 11 is within the body 128 of the housing 124, the body 128 may be placed over a target area of a user to be injected with the medicament contained within the container 11 in one embodiment. Once the housing 124 is placed over the target area of the user, the user may press downwardly on the deformable actuation mechanism component 126 to activate the container 11 such that any medicament within the container 11 is injected into the user. The body 128 of the housing 124 further includes an ejection mechanism 134 configured to eject the container 11 from the housing 124 when the ejection mechanism is activated.

In a further embodiment, a compressible component 136 can be provided on or associated with the lower surface 132 of the housing 124, such that when the housing 124 is placed onto the user and the compressible component 136 is compressed, a locking mechanism of the housing is released and the container 11 can be activated by the actuation mechanism component 126.

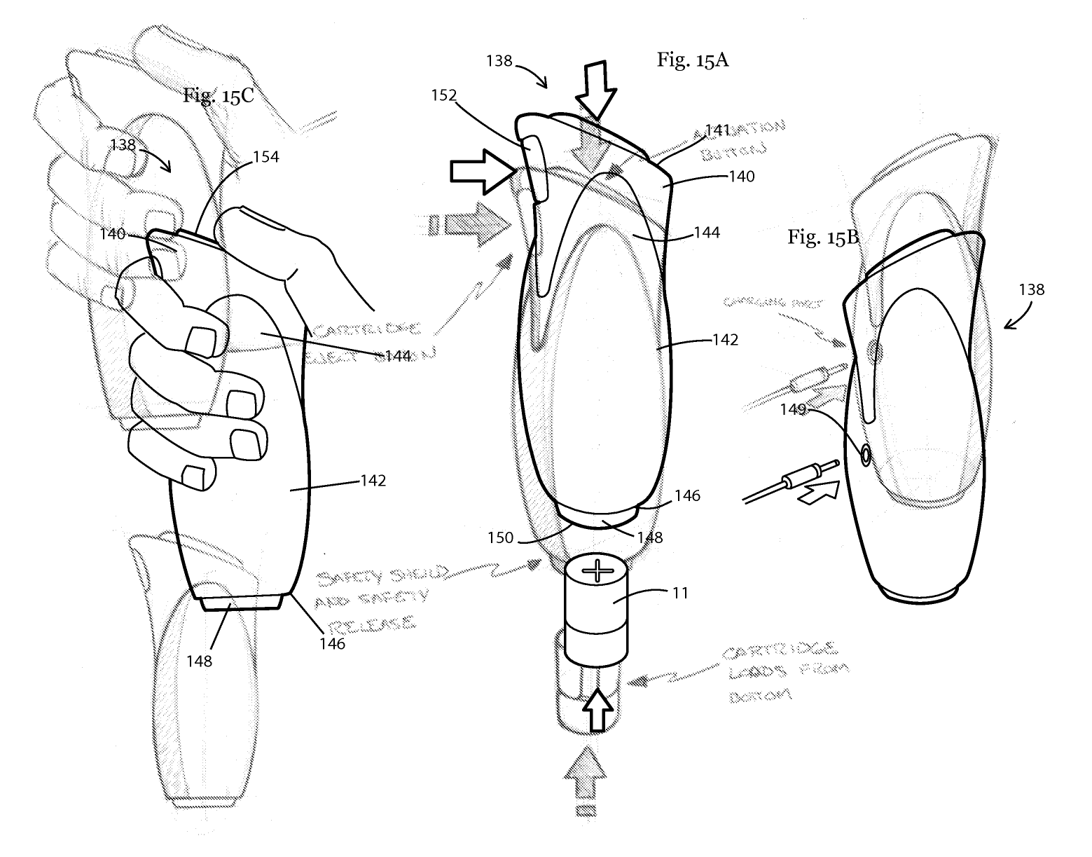

In still a further embodiment, another medicament delivery system housing 138 for use with a removable medicament container 11 is provided as shown in FIGS. 15A-C. The housing 138 includes a generally cylindrically shaped body portion 142 including a first end 144 and a second end 146, wherein the first and second ends 144, 146 have a smaller diameter than a middle section of the body portion 142, and the second end 146 includes an opening or socket 150 configured to receive a container 11. The housing embodiment 138 further includes a head portion 140 associated with the body portion 142, wherein the head portion 140 includes an upper surface 141 and a lower surface (not shown in FIG.). The head portion 140 associates with the body portion 142 at its lower surface, and an actuation mechanism 154 is disposed on its upper surface 141, the actuation mechanism 154 configured to activate the container 11. The head portion 140 further comprising a release member or ejection mechanism 152 configured to release the container 11 upon activation of the release member 152.

The housing embodiment 138 may further include a safety mechanism 148 disposed on the second end 146 of the body portion, wherein the safety mechanism 148 is released by pressing the second end 146 of the body portion 142 of the housing 138 or the safety mechanism 148 against a surface, wherein a release of the safety mechanism 148 allows the actuation mechanism 154 to be activated and the container 11 to be activated. In one embodiment, pressing the safety mechanism 148 against a surface may cause it to move toward the body portion 142 and enable use of the device. An opening 149 may be provided on the housing 138 as shown in FIG. 15B such that a power source provided within the medicament delivery system housing 138 can be re-charged by connection to an electrical outlet, battery, or other rechargeable mechanism between uses.

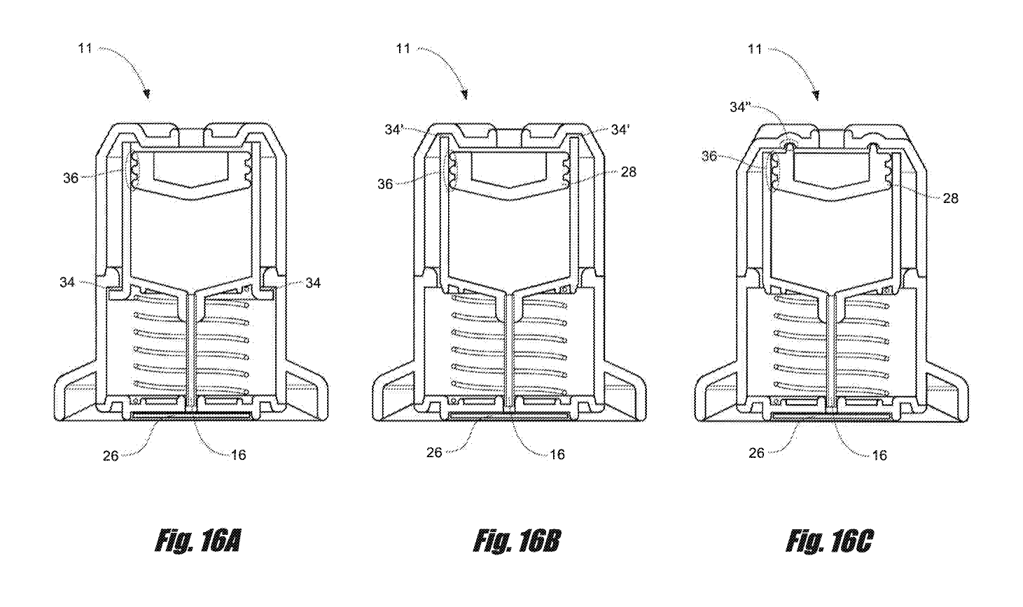

FIGS. 16A-C provide container 11 embodiments with varying second contaminant barriers. FIG. 16A provides a second contaminant barrier 34 between the first housing 10 and the second housing 12, and FIG. 16B provides second contaminant barriers 34′ between an upper portion of the first housing 10 and the second housing 12. FIG. 16C provides a second contaminant barrier 34″ as part of the stopper 28, such that the second contaminant barrier 34″ associates with the second housing 12 until the stopper 28 is moved relative to the first housing 10 in a first direction to deliver the medicament through the injection member 16. Other contaminant barriers such as the third contaminant barrier 36 and the first contaminant barrier 26 are shown in FIGS. 16A-C.

FIG. 17A provides a cross sectional view of a container embodiment 11′ alternative to FIG. 16B. FIG. 17B-C provide an exploded view of the container embodiment 11′ wherein the first portion of the second housing 12A extends from the bottom of the container 11′ to the top of the container, and encases the first housing 10, and the second portion of the second housing 12B can be attached to the first portion 12A of the second housing. The second contaminant barriers 34′ are shown in FIG. 17A as well as the first contaminant barrier 26. FIG. 17C shows the first housing 10 with injection member 16 and the stopper 28. The stopper 28 may also include a soft silicone outer portion and a hard stopper core 29, for example, as shown in the non-limiting embodiment of FIG. 17C. The spring 30 in the embodiment of FIG. 17A is shown as disposed between the upper portion of the first housing 10 and the lower portion of the second housing 12.

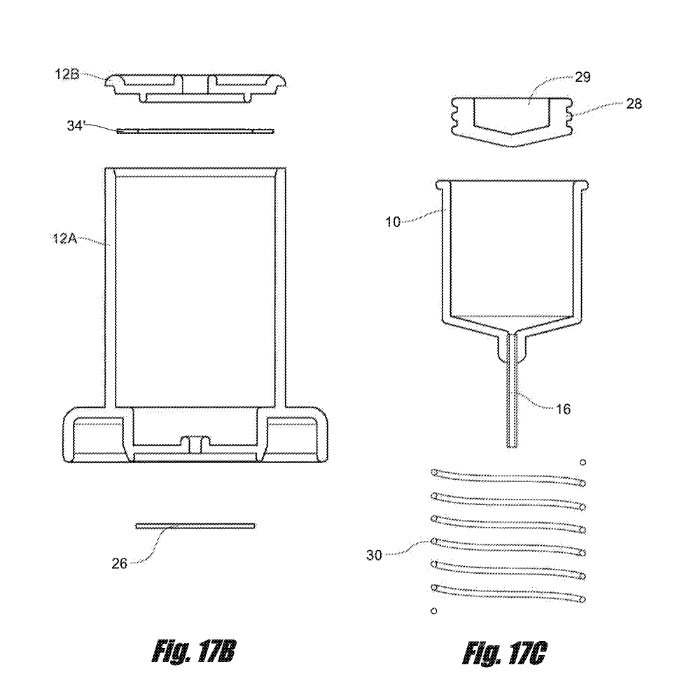

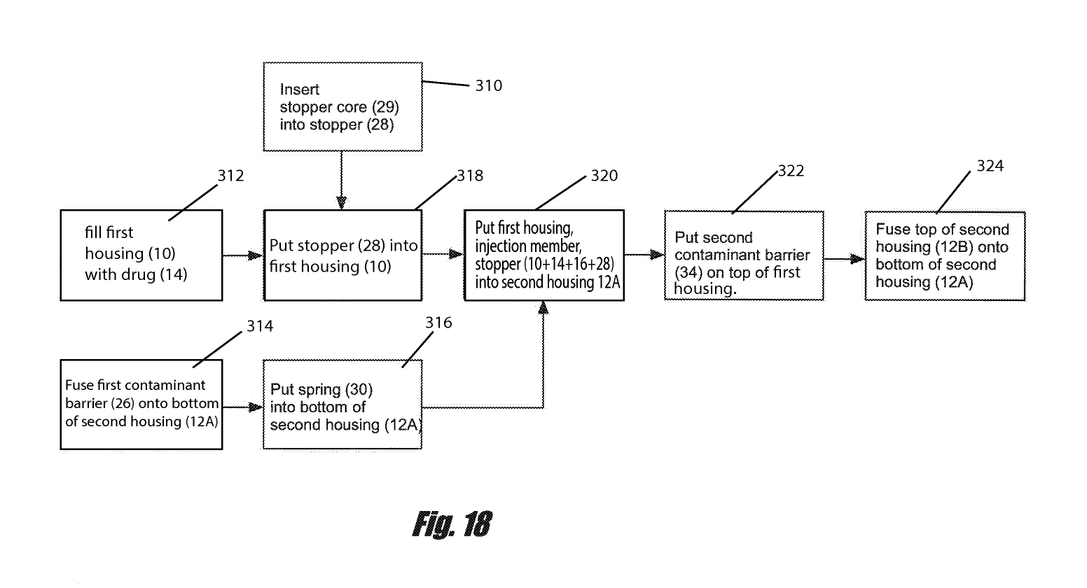

FIG. 18 is a block diagram of a flow chart demonstrating a non-limiting example of a method of manufacturing the container embodiment of FIG. 17A, wherein the components of the container embodiment 11′ of FIG. 17A are shown in the exploded view of container 11′ in FIGS. 17B, 17C. In this non-limiting method embodiment, a stopper core can be inserted into a stopper 310, a first housing can be filled with a medicament 312, and a first contaminant barrier can be attached or fused to the bottom of the second housing 314. A spring can be placed into the bottom of the second housing 316, the stopper can be placed into the first housing 318, and the first housing with the medicament, injection member and stopper can be placed into the second housing 320, the second contaminant barrier can be placed on top of the first housing 322, and the top of the second housing can be fused onto the bottom of the second housing 324, in a non-limiting embodiment.

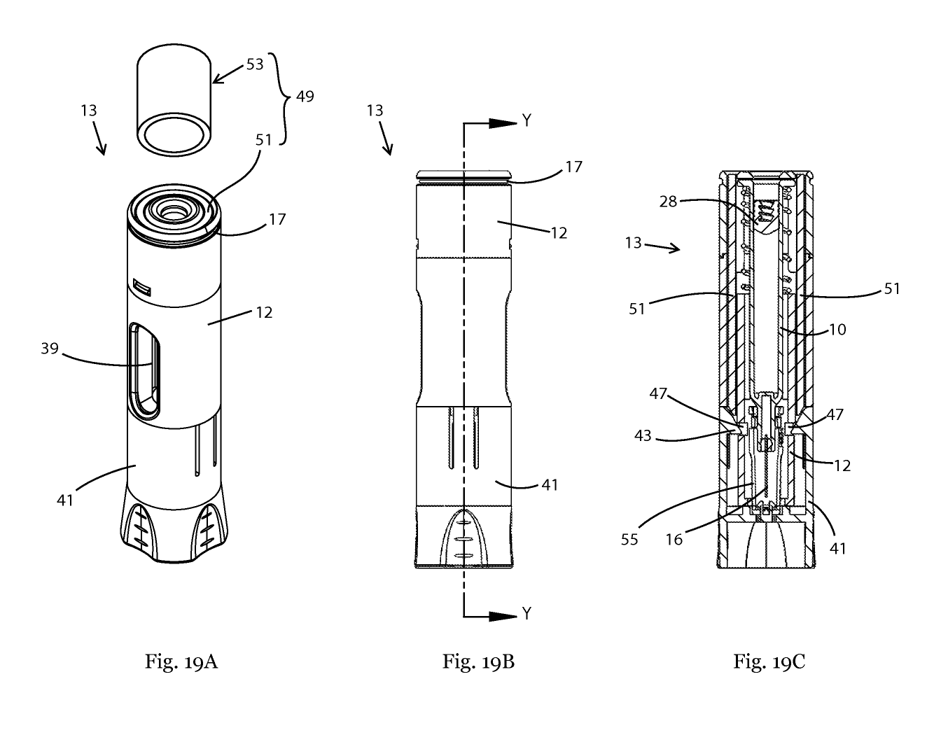

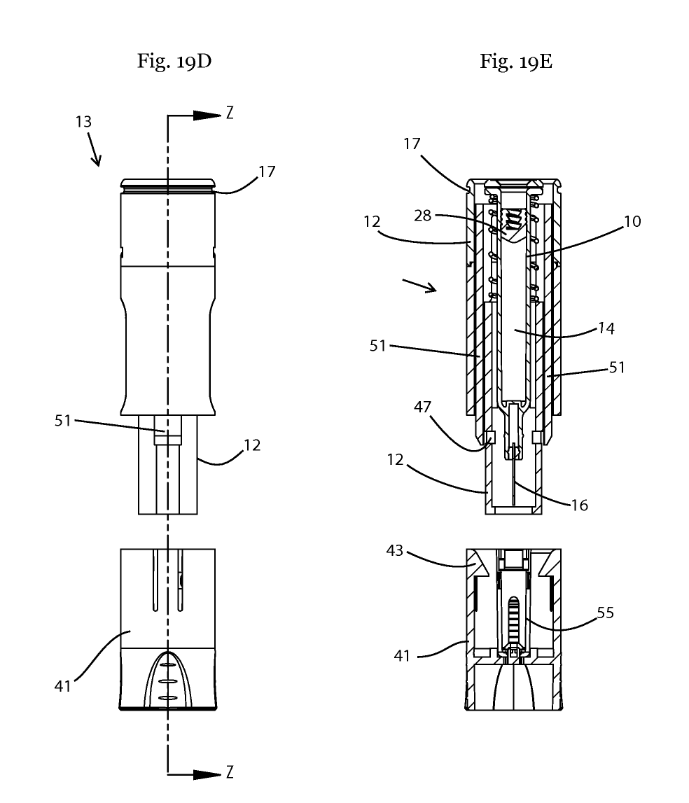

FIG. 19A is a perspective view of an embodiment of a container 13 having a first housing 10 disposed within a second housing 12, wherein the second housing 12 has an indented portion 17 at an upper portion thereof. The second housing may further include openings or windows 39 providing a view of the medicament held within the first housing 10 there through. The container 13 further includes a cover 41 associated with a lower portion thereof. The cover 41 may be associated with either the first housing 10 or the second housing 12, or a combination thereof. The cover 41 may include one or more protrusions 43 on an inner surface thereof which may engage with one or more notches 47 which may be provided on an outer surface of either the first housing 10 or the second housing 12 allowing the cover 41 to be engaged to the first or second housing 10, 12. FIG. 19B is a side elevational view of the container 13 embodiment shown in FIG. 19A. The cover 41 may further include or be associated with an injection member cap 55. The injection member cap 55 is configured to cover the injection member 16 prior to use of the container 13. The injection member cap 55 may be affixed to a portion of the cover 41 such that when the cover 41 is removed from the container 13, the injection member cap 55 is removed from the injection member 16 prior to use of the container 13. This removal of the cover 41 and injection member cap 55 may occur once the container 13 is within a reusable housing, in a non-limiting embodiment, to provide protection to a user preventing an unwanted stick with the injection member 16.

In a further embodiment, the container 13 may include a releasing mechanism 49 which may include a releasing member 51 and a releasing member actuator 53 in a non-limiting embodiment. The releasing member 51 may be disposed within the second housing 12 of the container 13 as shown in the cross sectional view of FIG. 19C taken along line Y-Y of FIG. 19B. Exerting a force on the releasing member 51 in a direction toward the cover 41 may cause the one or more protrusions 43 to disengage from the one or more notches 47, such that the cover 41 is released from the container 13. This force exertion on the releasing member 51 may be accomplished by way of a releasing member actuator 53, in a non-limiting embodiment, which may be a separate component in one non-limiting embodiment, or may be part of the reusable housing component in another non-limiting embodiment within which the container 13 may be received.

FIG. 19D shows a side elevational view of the container 13 embodiment with the cover 41 removed there from. The indented portion 17 and the second housing 12 of container 13 are shown in FIG. 19D. FIG. 19E is a cross sectional view of the container 13 embodiment of FIG. 19D taken along line Z-Z showing the first housing 10, the second housing 12, the notches 47 on the second housing 12, the injection member 16 extending from the first housing 10, and the medicament 14 and stopper 28 within the first housing 10. FIG. 19E also shows spring 30 disposed between the first housing 10 and second housing 12. A cross sectional view of the cover 41 with protrusions 43 on an inner surface thereof, and an injection member cap 55 retained within the cover 41 is provided in FIG. 19E.

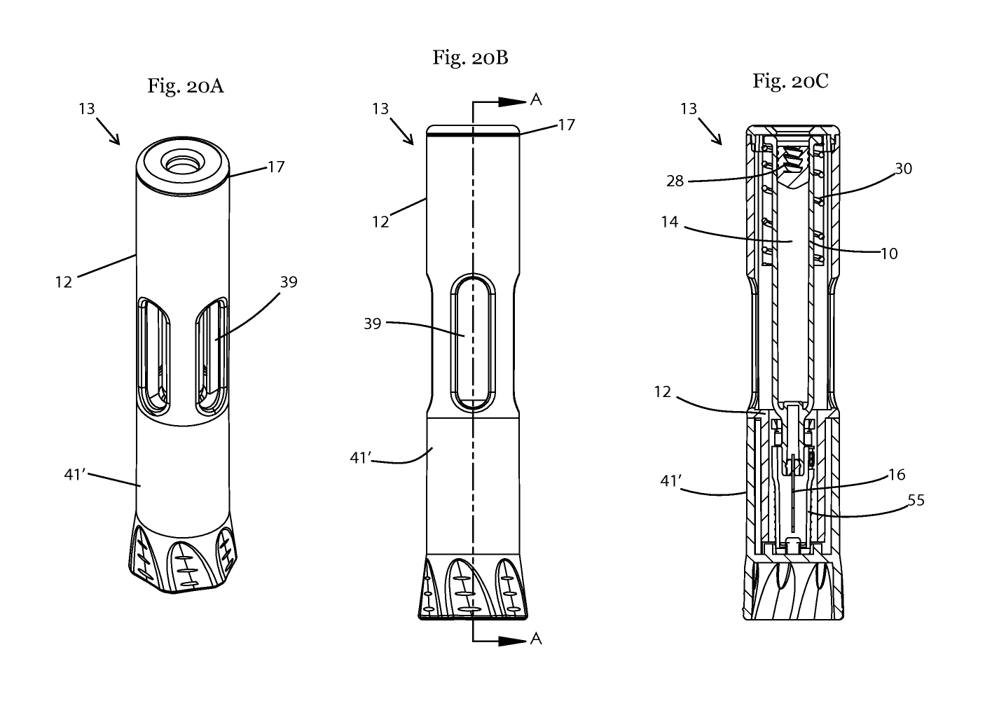

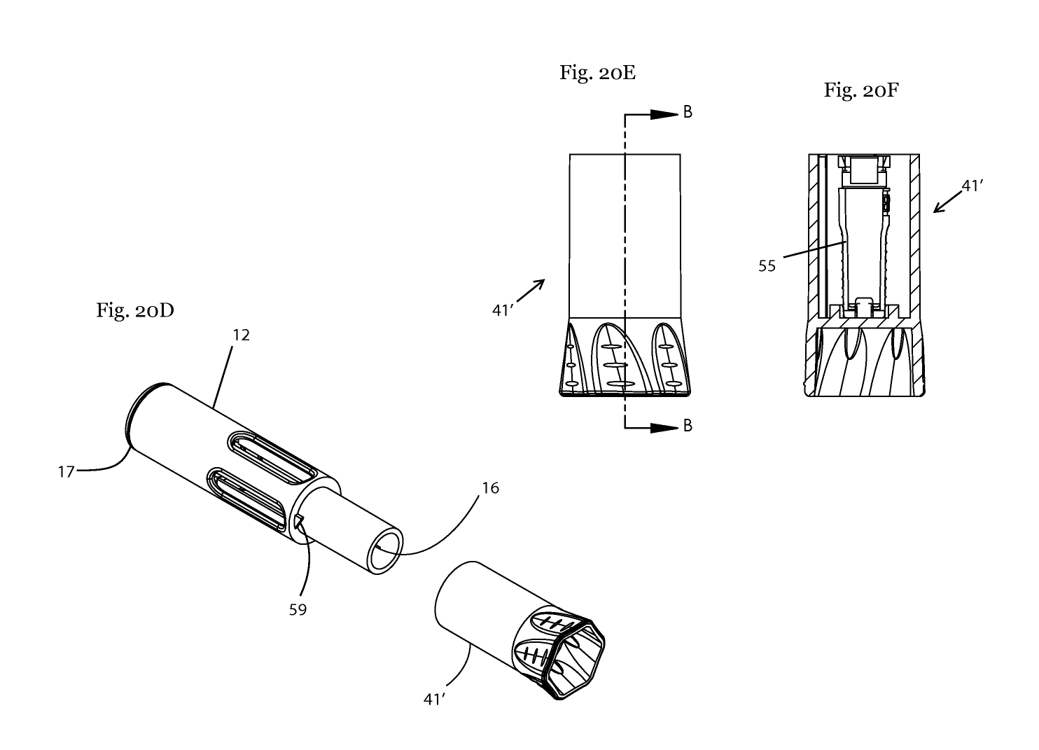

FIGS. 20A-F include various views of a non-limiting embodiment of container 13 wherein a cover 41′ is removably coupled thereto. FIG. 20A is a perspective view of container 13 having indented portion 17 around the upper portion of housing 12, and windows or openings 39 allowing a view of the amount of medicament remaining within the first housing 10. A cover 41′ is engaged with the second housing 12 of the container 13. Movement of the first housing 10 relative to the second housing 12 to eject the injection member 16 and deliver medicament can occur via an actuation mechanism as described herein in non-limiting embodiments. The actuation mechanism may activate the container 13 via an actuation aperture 57, in one non-limiting embodiment.

FIG. 20B is an elevational view of the non-limiting embodiment of the container 13 of FIG. 20A showing a second housing 12 with a window 39 and cover 41′ attached thereto. FIG. 20C is a cross sectional view of the container 13 shown in FIG. 20B, wherein a spring 30 is disposed between the first housing 10 and the second housing 12, and a stopper 28 and medicament 14 is housed within the first housing 10. An injection member 16 extends from a lower portion of the first housing 10, wherein an injection member cap 55 is shown surrounding the injection member 16 in a non-limiting embodiment. The injection member cap 55 is associated with an inner portion of the cover 41′ in the non-limiting embodiment shown in FIG. 20C. The injection member cap 55 may be affixed to a portion of the cover 41′.

FIG. 20D is a perspective view of the container 13 of FIGS. 20A-C, wherein the cover 41′ has been removed from the second housing 12 exposing the injection member 16 contained within the walls of the second housing 12. In the non-limiting embodiment shown in FIGS. 20A-F, the cover 41′ may be manually removed from the second housing 12 by a user. The cover 41′ may be retained on the second housing 12 by a snug fit between the components, in one non-limiting example. Other non limiting examples may include engagement between mechanical components known to those skilled in the art that allow manual removal of the cover 41′ by a user. For example, a first mechanical component 59 on the second housing 12 may engage with a second mechanical component 61 (not shown) on the cover 41′ such that disengagement of the first and second mechanical components 59, 61 may allow removal of the cover 41′ from the container 13. This removal may require a simple turn of the cover 41′ to disengage the cover 41′ from the container 13 in one non-limiting embodiment.

FIG. 20E is a side elevational view of an embodiment of a cover 41′. FIG. 20F is a cross sectional view of the cover 41′ embodiment shown in FIG. 20E, taken at line B-B. The injection member cap 55 is shown as disposed within the cover 41′ in FIG. 20E.

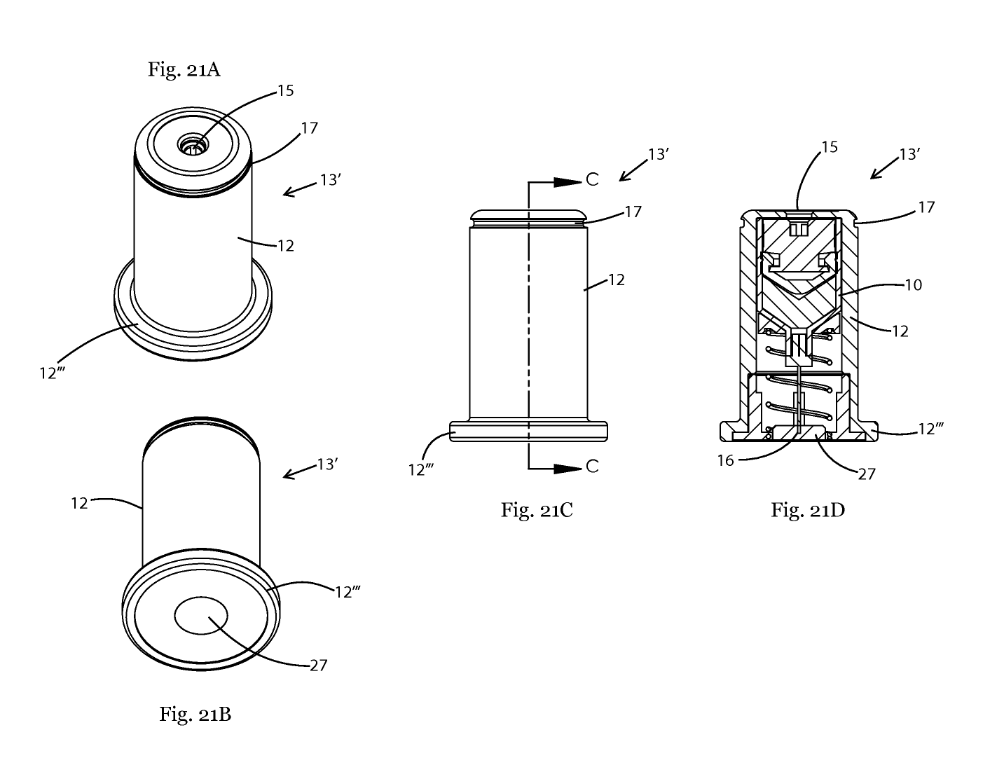

FIGS. 21A-D are views of a further embodiment of a container 13′ including an actuation aperture 15, a first housing 10 disposed within a second housing 12, the second housing having an indented portion 17 around its circumference on an upper portion thereof. The indented portion 17 may surround the circumference of the second housing 12 as shown in FIGS. 21A-D, or may be provided on only a portion of the second housing 12 in an alternative embodiment (not shown). Some non-limiting embodiments of the container shown throughout the disclosure include a base portion including a larger diameter than the rest of the body of the container. The embodiment of the container 13′ shown in FIGS. 21A-D include a base portion 12′″ of second housing 12 which is larger in diameter than the remainder of the second housing 12. This base portion 12′″ is provided, for example, such that the container 13′ can be stable when positioned on a surface, and may also aid in positioning the container within a reusable housing, when the container is disposed therein, such that the base portion 12′″ may abut a lower portion of the reusable housing so as to prevent the container 13′ from being inserted too far into the reusable housing, in a non-limiting embodiment.

In the non-limiting container embodiment 13′ shown in FIGS. 21B and 21D, a portion of the bottom surface of the container 13′ may include a septum barrier 27 which serves to protect the injection member and the inside of the second housing 12 from below to maintain sterility of the container 13′ and prevent contamination of the injection member 16 and the inner portion of the second housing 12 of the container 13′. The injection member 16 may traverse the septum barrier 27 during use of the container 13 when the injection member 16 is delivered from the second housing 12. The septum barrier 27 may include a rubber septum material in one, non-limiting embodiment. The septum barrier may include any materials traversable by an injection member in a non-limiting embodiment. Other materials known to those skilled in the art may be used to form the septum barrier. The septum barrier 27 is shown in FIG. 21B as forming a circular portion in the center of the base portion 12′″ of the second housing 12; however, the septum barrier 27 is not limited in shape or size to the embodiment shown in FIG. 21B. The entire bottom portion of the second housing 12 may include the septum barrier 27 in another non-limiting embodiment, and in still a further non-limiting embodiment, the septum barrier 27 may form any portion of the of the second housing 12. In a further non-limiting embodiment, the septum barrier 27 may form all or part of the second housing 12. FIG. 21D shows across sectional view of the container embodiment 13 taken along line C-C in FIG. 21C.

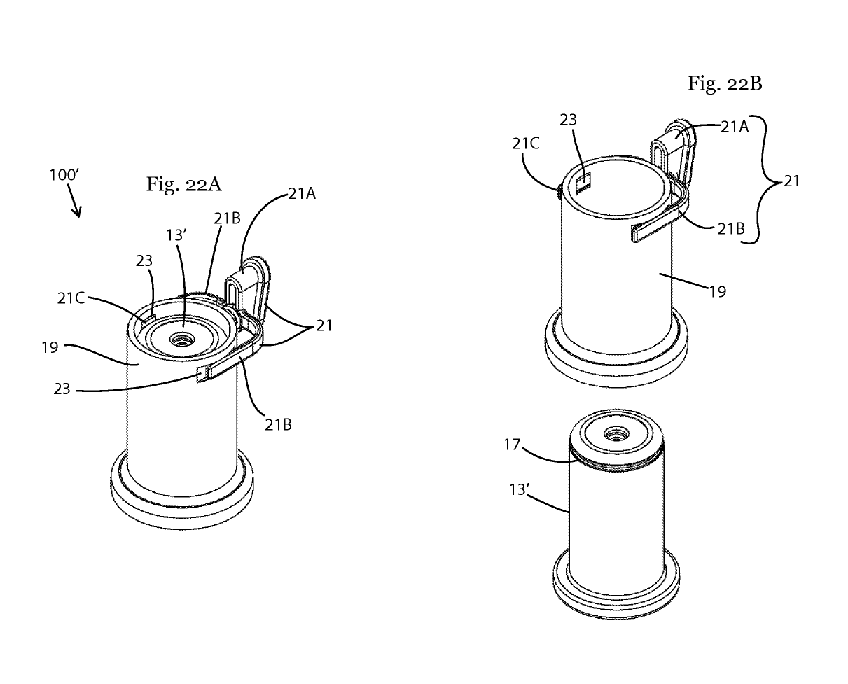

FIG. 22A-22B are perspective views of a portion of an embodiment of the medicament delivery training system 100′ wherein a shell portion 19 is disposed within a reusable housing component (not shown) of the system 100′. The shell portion 19 is configured to receive and retain the container 13′ when the container 13′ is inserted into the reusable housing component. The shell portion 19 or the reusable housing component includes a triggering mechanism 21 which includes a triggering element 21A and one or more locking elements 21B. The shell portion 19 may include one or more apertures 23 in its outer wall. The one or more apertures 23 are configured to align with the indented portion 17 of the container 13′ when the container 13′ is placed correctly within the shell portion 19. Each of the one or more locking elements 21B of the triggering mechanism 21 include a protruded portion 21C, which is provided to engage the indented portion 17 of the container 13′ through the one or more apertures 23 of the shell portion 19 to retain the container 13′ within the reusable housing when the container 13′ is placed correctly within the shell portion 19. The triggering element 21A is associated with the locking element(s) 21B of the triggering mechanism 21, such that actuation of the triggering element 21A moves the protruded portion(s) 21C of the locking element(s) 21B away from the aperture(s) 23 to disengage the locking element(s) 21B from the indented portion 17 of the container 13 to release the container 13′ from the shell portion 19 and from the reusable housing after use. FIG. 22B is a perspective view of the container 13′ once it has been released from the shell portion 19.

In one non-limiting embodiment, the shell portion 19 and/or the triggering mechanism 21 may be provided as part of the reusable housing component of the system 100′ (i.e., disposed within the reusable housing component). In another non-limiting embodiment, the shell portion 19 and/or the triggering mechanism 21 may be associated with the container 13′ during transport of the container 13′, for example. The embodiment of FIG. 22A-B including the shell portion 19, triggering mechanism 21 and other components may further be used with other container embodiments described herein.

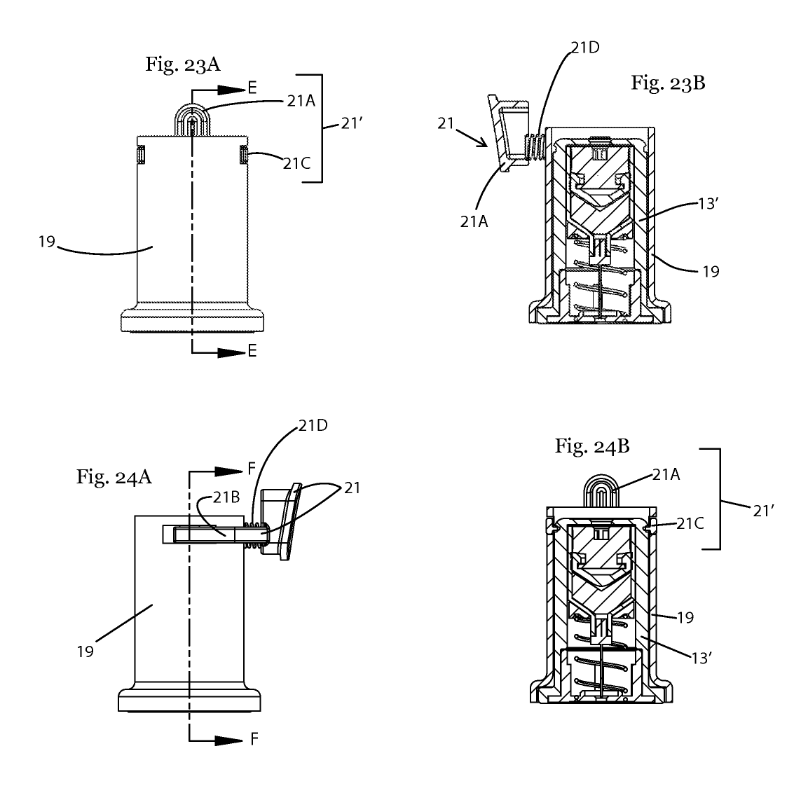

FIG. 23A is a front elevational view of a shell portion 19 with a container 13′ disposed there within. FIG. 23B is a cross sectional view of the shell portion 19 and container 13′ taken along line E-E of FIG. 23A. FIG. 24A is a right side elevational view of the shell portion 19 embodiment shown in FIG. 23A. FIG. 24B is a cross sectional view of the shell portion 19 embodiment of FIG. 24A taken along line F-F of FIG. 24B. FIGS. 23A-B and 24A-B include a triggering mechanism embodiment 21′ including a triggering spring component 21D disposed between the triggering element 21 and the shell portion 19. The triggering spring component 21D may allow the triggering element 21A to reset to its starting position after it is pressed to release the container 13′.

The triggering element 21A as described herein may be provided, in a non-limiting embodiment, on an outer surface of the reusable housing component of the system 100′, wherein they are accessible by a user for release of the container 13′ from the reusable housing component upon actuation of the triggering element 21A. The triggering mechanism embodiment 21′ and/or the shell portion 19 may be used with other container embodiments described herein in non-limiting embodiments and is not limited to use with the container 13′ shown in FIGS. 23A-24B.

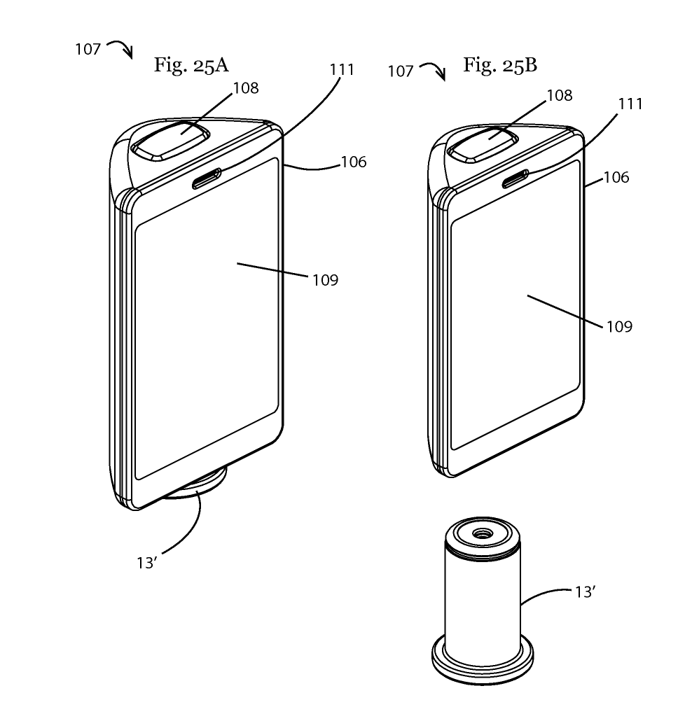

FIG. 25A is a perspective view of a reusable housing component embodiment 107, the reusable housing embodiment 107 may include a reusable housing component 106 which may resemble a Smartphone in a non-limiting embodiment. In another non-limiting embodiment, the reusable housing component 106 may include a Smartphone. Other electronic devices include a PDA, a computer, and other smart devices may be used as the reusable housing component 106, the Smartphone embodiment shown is provided only as an example and is not intended to be limiting. The reusable housing component 106 may include an actuation member 108 disposed on a portion thereof, the actuation member 108 may be associated with an actuation mechanism disposed within the housing 106. The reusable housing component embodiment 106 may be configured to receive a container 13, 13′, 11 as shown in FIG. 25A (container embodiment 11, 13 not shown in FIGS. 25A, B) for delivery of a medicament and/or to train a user for a medicament delivery. The actuation mechanism 108 may be provided to activate the container 13, 13′ 11, to deliver medicament to a user in a manner described herein or to train a user to deliver a medicament. A display 109 may be provided on the reusable housing component embodiment 106 as shown in FIGS. 25A, 25B to provide information to the user and/or receive information from a user via virtual buttons, in a non-limiting example. Further optional features may be provided on the reusable housing component 106 including, but not limited to, responsive members reactive to user input (e.g., buttons), a speaker 111 to provide an audible output to a user, one or more lights, tactile components, smell emitting components, lights or other visual stimuli, or any other components known to one skilled in the art to provide feedback and/or information to a user.

FIG. 25B provides a perspective view of the reusable housing component embodiment 106 of FIG. 25A including actuation mechanism 108, speaker 111, and display 109, with the container 13′ removed there from. The reusable housing component embodiment 106 may further include the triggering mechanism embodiments 21, 21′ as well as other embodiments of other housing components and/or other systems as described herein.

In a further embodiment, a medicament delivery system is provided, the system includes a container for storing a medicament prior to use. The container includes a first housing for containing a medicament a stopper associated with the first housing, a second housing, wherein the first housing is held within the second housing, and the first housing is movable relative to the second housing. The container further includes an injection member associated with a lower portion of the first housing. The system further includes an actuation mechanism, wherein the actuation mechanism interacts with the container to move the first housing relative to the second housing in a first direction to eject the injection member from the second housing, traversing a first contaminant barrier, and move the stopper relative to the first housing in a first direction to deliver the medicament through the injection member, such that when the first housing moves relative to the second housing in a second direction, the injection member is retracted into the second housing to prevent an unintentional contact with the injection member.

In yet a further embodiment, a container for storing a medicament prior to use is provided. The container may include a first housing for containing a medicament, a stopper associated with the first housing, a second housing, wherein the first housing is held within the second housing, and the first housing is movable relative to the second housing, and an injection member associated with a lower portion of the first housing. An actuation mechanism is configured to interact with the container to move the first housing relative to the second housing in a first direction to eject the injection member from the second housing, traversing a first contaminant barrier, and move the stopper relative to the first housing in a first direction to deliver the medicament through the injection member, such that when the first housing moves relative to the second housing in a second direction, the injection member is retracted into the second housing to prevent an unintentional contact with the injection member.

It should be borne in mind that all patents, patent applications, patent publications, technical publications, scientific publications, and other references referenced herein are hereby incorporated by reference in this application in order to more fully describe the state of the art to which the present invention pertains.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

1. A medicament delivery system, comprising:
a container for storing a medicament prior to use, the container having one or more openings therein, the container comprising;
a first housing for containing a medicament;
a stopper associated with the first housing;
a second housing, wherein the first housing is held within the second housing, and the first housing is movable relative to the second housing;
an injection member associated with a lower portion of the first housing;
a spring disposed between the first housing and the second housing; and an actuation mechanism;
wherein the actuation mechanism interacts with the container to move the first housing relative to the second housing in a first direction, such that the spring is in a biased state, the injection member is ejected from the second housing traversing a first contaminant barrier, and move the stopper relative to the first housing in a first direction to deliver the medicament through the injection member, such that when the first housing moves relative to the second housing in a second direction, the spring is in an unbiased state and the injection member is retracted into the second housing to prevent an unintentional contact with the injection member.

2. The medicament delivery system of claim 1, wherein the second housing surrounds at least a portion of the first housing to prevent an unintentional actuation of the container, said second housing comprising at least one opening configured to receive the actuation mechanism.

3. The medicament delivery system of claim 1, wherein the actuation mechanism is associated with the stopper, such that upon activation of the actuation mechanism, the first housing moves relative to the second housing in a first direction to eject the injection member, and the stopper moves relative to the first housing in a first direction to deliver the medicament through the injection member, wherein, optionally, the first housing displacement component comprises one or more projections and the medicament delivery system further comprises a barrel actuation mechanism, said barrel actuation mechanism configured to surround at least a portion of the container, the barrel actuation mechanism comprising an outer portion comprising one or more nodules and an upper surface comprising one or more grooves, said upper surface of the barrel actuation mechanism configured to interact with the one or more projections on the first housing displacement component, wherein said barrel actuation mechanism is rotatable relative to the container.

4. The medicament delivery system of claim 1, wherein the container is configured to interact with a first housing displacement component wherein said first housing displacement component comprises one or more projections, wherein the one or more projections are configured to interact with the first housing through the one or more openings of the second housing to move the first housing in at least a first direction relative to the second housing when the first housing displacement component is activated.

5. The medicament delivery system of claim 1, wherein said container is configured to interact with a stopper displacement component, said stopper displacement component comprises one or more projections, wherein the one or more projections of the stopper displacement component are configured to interact with the stopper through the one or more openings of the second housing to move the stopper relative to the first housing in at least a first direction when the stopper displacement component is activated.

6. The medicament delivery system of claim 3, further comprising a second spring adjacent to the first housing displacement component, said second spring configured to activate the first housing displacement component when the second spring is compressed to move the first housing relative to the second housing in the first direction to displace the injection member from the second housing.

7. The medicament delivery system of claim 5, further comprising a first actuation mechanism, said first actuation mechanism configured to activate the stopper displacement component to move the stopper relative to the first housing in the first direction to deliver medicament through the injection member when the first actuation mechanism is activated.

8. The medicament delivery system of claim 7, wherein the first actuation mechanism comprises a solenoid.

9. The medicament delivery system of claim 3, further comprising a second actuation mechanism, said second actuation mechanism configured to activate the first housing displacement component to move the first housing relative to the second housing in the first direction to displace the injection member from the second housing.

10. The medicament delivery system of claim 9, wherein the second actuation mechanism comprises a solenoid.

11. The medicament delivery system of claim 1, further comprising a second contaminant barrier between the upper portion of the second housing and a lower portion of the first housing and/or between the upper portion of the first housing and the second housing to prevent contaminants from entering the second housing.

12. The medicament delivery system of claim 1, further comprising a third contaminant barrier between the stopper and the first housing to prevent contaminants from entering the first housing.

13. The medicament delivery system of claim 1, wherein a first actuation mechanism is configured to interact with a stopper displacement component, wherein said stopper displacement component is configured to interact with the stopper such that when a first actuation mechanism is activated, the first housing moves in a first direction relative to the second housing, the injection member is delivered from the second housing, and the stopper moves in a first direction relative to the first housing to deliver medicament from the first housing through the injection member.

14. The medicament delivery system of claim 13, wherein the first actuation mechanism comprises a solenoid.

15-19. (canceled)

20. The medicament delivery system of claim 3, further comprising a first housing displacement component configured to interact with the first housing to move the first housing in at least a first direction relative to the second housing when the first housing displacement component is activated is associated with a sleeve, said sleeve comprising one or more sleeve nodules on a portion thereof.

21. The medicament delivery system of claim 20, wherein the second housing comprises one or more openings, and wherein said first housing displacement component comprises one or more projections configured to contact the first housing through the one or more openings of the second housing.

22. The medicament delivery system of claim 21, further comprising a motor, said motor having one or more nodules, wherein said motor nodules are complementary to said sleeve nodules such that the motor nodules and the sleeve nodules interact with one another when the motor is actuated to control the movement of the first housing displacement component relative to the container to move the first housing relative to the second housing.

23. The medicament delivery system of claim 1, wherein a contact sensor is associated with the container, said contact sensor configured to detect contact between the sensor and a user, wherein an output signal is provided to a processor based on the contact detected by the contact sensor.

24. The medicament delivery system of claim 23, wherein the processor is associated with the stopper and a motor associated with either the first housing and/or the stopper, such that when the contact sensor detects no contact between the patient and the contact sensor, the processor stops the motor from moving the first housing in a first direction relative to the second housing and/or the stopper in a first direction relative to the first housing to prevent the injection member from being delivered from the second housing and/or the medicament from being delivered through the injection member.

25. A medicament delivery training system configured to provide stepwise instructions for using the system to a user in a particular sequence, the system comprising:
a reusable housing component configured to receive a container, said reusable housing component comprising a control interface, the control interface comprising at least one responsive member reactive to a user input;
an actuation mechanism configured to interact with the container;
a signal output component associated with the reusable housing component; and
circuitry associated with the reusable housing component configured to control a provision of the stepwise instructions to the user in the particular sequence, wherein the container comprises a first housing, and a second housing, a spring disposed between a lower portion of the first housing and a lower portion of the second housing, and an injection member associated with the lower portion of the first housing, wherein the first housing is movable relative to the second housing; and a stopper is associated with the first housing, said stopper being movable relative to the first housing, wherein the actuation mechanism interacts with the container to move the first housing in a first direction relative to the second housing to eject the injection member from the second housing, and the stopper in a first direction relative to the first housing.

26. The medicament delivery training system of claim 25, further comprising wherein the first housing comprises a medicament to be delivered to a patient, such that when the stopper moves relative to the first housing in a first direction, medicament is delivered through the injection member.

27. The medicament delivery training system of claim 25, comprising a training mode and/or a medicament delivery mode.

28. The medicament delivery training system of claim 27, wherein a provision of stepwise instructions for using the system is provided to the user in the training mode before the medicament delivery mode can be used.

29. The medicament delivery training system of claim 25, further comprising a locking mechanism associated with the actuation mechanism, such that the actuation mechanism cannot be activated until the locking mechanism is released, wherein said locking mechanism is released upon completion of the stepwise instructions for using the system.

30. The medicament delivery training system of claim 25, wherein the actuation mechanism cannot be activated to interact with the container until the stepwise instructions for using the system have been provided to the user.

31-55. (canceled)
